(19) United States
(12) Patent Application Publication
(10) Pub. No.: US 2024/0245703 A1
(43) Pub. Date: Jul. 25, 2024

(54) TOPICAL APPLICATION OF AGENTS TO REDUCE SUN SENSITIVITY AND IMPROVE TOPICAL PHOTODYNAMIC THERAPY

(71) Applicants: Wright State University, Dayton, OH (US); TheUnited States of America as represented by the Department of Veterans Affairs, Washington, DC (US)

(72) Inventor: Jeffrey B. TRAVERS, Centerville, OH (US)

(21) Appl. No.: 18/623,530

(22) Filed: Apr. 1, 2024

Related U.S. Application Data
(63) Continuation-in-part of application No. PCT/IB2022/059372, filed on Sep. 30, 2022.
(60) Provisional application No. 63/250,654, filed on Sep. 30, 2021.

(51) Int. Cl. A61K 31/55; A61K 41/00; A61K 45/06; A61P 17/16

(52) U.S. Cl. CPC A61K 31/55; A61K 41/0061; A61K 45/06; A61P 17/16

(57) ABSTRACT

A skin protective composition includes at least one functional inhibitor of an acid sphingomyelinase (FIASM) in a dermatologically acceptable carrier. A method of ameliorating photosensitivity in a subject includes topically administering the skin protective composition to a subject, where the therapeutically effective amount is demonstrated to reduce or eliminate inflammation, microvesicle particle release, erythema, or a combination thereof. In another embodiment, a method inhibits the release of microvesicle particles from skin cells. In another embodiment, a method reduces side effects resulting from a topical photodynamic therapy treatment.

### REFERENCE TO RELATED APPLICATIONS

This application is a continuation-in-part of International Application No. PCT/IB22/59372, filed Sep. 30, 2022, which claims priority to and the benefit of U.S. Provisional Application No. 63/250,654, filed Sep. 30, 2021, both of which are hereby incorporated by reference in their entirety.

### GOVERNMENT RIGHTS

This invention was made with government support under ES031087 and HL062996 awards by the National Institutes of Health, and 510BX000853 award from the US Veterans Administration. The government has certain rights in the invention.

### FIELD OF THE DISCLOSURE

The instant disclosure is directed to methods and compositions for providing protective care to skin employing a topically applied functional inhibitor of acid sphingomyelinase for prophylaxis and relief from the effects of photosensitivity resulting from one or more of exposure to ultraviolet (UV) radiation and light exposure attendant to photodynamic therapy.

### BACKGROUND

Photosensitivity is the clinical term for abnormal reactions to ultraviolet radiation (e.g., sunlight). Several disease states including lupus erythematosus and rosacea exhibit photosensitivity. Photosensitivity is a heightened skin sensitivity to UV radiation. There are multiple causes of photosensitivity, including side effects from certain medications, use of certain skincare products, or as a part of a more extensive medical condition or genetic disorder. Photosensitivity can be either photoallergic or phototoxic. Phototoxicity is the more common type of photosensitivity reaction and can occur when a drug that has been taken is activated by UV light. This causes damage to the skin that can appear similar to a rash or sunburn. Photoallergic reactions happen when UV light interacts directly with something applied directly to the skin, causing the immune system to recognize the activated materials. Certain diseases also increase the risk for photosensitivity reactions. These include lupus erythematosus, pellagra, psoriasis, rosacea, polymorphous light reactions, and xeroderma pigmentosum (XP). A common model for photosensitivity for both cells and mice involves loss of the XP complementary group A (XPA) protein. Photosensitivity is painful and can increase the risk of skin cancer. It should be noted that in certain diseases such as lupus erythematosus, photosensitive reactions can trigger systemic inflammation including lung and kidney damage.

These types of reactions are treated similarly to caring for a sunburn, such as, for example, with one or more of salves and pain and anti-inflammatory medication. Prevention methods include minimizing exposure to the sun, using hats or sunscreen when in the sun, closely observing the skin, and visiting a dermatologist regularly. While in many cases, doctors can diagnose the condition by just looking at the skin and considering an individual's medical history, occasionally specialized tests are needed. These can include UV light testing, photo patch testing, and testing the blood or skin with a biopsy. Prevention and care for photosensitivity depends on the severity of the reaction. While the symptoms resolve with a break from direct sunlight in many cases, anti-inflammatory medications may be necessary in more severe cases, either applied topically or taken systemically. In more severe cases, additional protective steps may include the use of special UV-blocking films on windows. While these conditions are not life-threatening, they can cause significant suffering.

Relatedly, photosensitivity and its attendant challenges and risks can be present in the context of photodynamic therapy. Topical photodynamic therapy (PDT) is a treatment that includes topically applying a photosensitizing agent, which is absorbed by cells, and the treatment site is then exposed to a light source such as a laser or a blue/red light-emitting diode after a pre-determined amount of time. The clinical uses of PDT include multiple neoplastic indications, skin disorders, and ocular conditions. When the light is applied at a specific wavelength to the targeted area, the photosensitizing agent undergoes a reaction that forms one or more reactive oxygen species (ROS) that kills the targeted cells and/or induces vascular damage. Although PDT it can be effective and beneficial in treating actinic damage, including tumors and pre-cancerous lesions, PDT has side effects, including pain, redness, and photosensitivity.

There is strong evidence to suggest that PDT causes local immunosuppressive side effects. Immunosuppression is an important risk factor for skin cancer development. The immunosuppressive effects of PDT are thought to be responsible for reports of more aggressive tumors growing in PDT-treated areas, as well as the lack of response of precancerous lesions to this treatment modality. The side effects of discomfort and lack of efficacy impact the overall success of PDT. Efforts to mitigate the resulting immunosuppressive effects could help improve efficacy or expand indications for PDT, but the mechanism of these immunosuppressive side effects is not well understood and no effective mitigation of these immunosuppressive side effects is available.

In view of the foregoing challenges with photosensitivity and photosensitivity specially in the context of PDT, interventions are needed to provide protection from photosensitivity.

### SUMMARY OF THE INVENTION

As disclosed herein, the inventors have unexpectedly demonstrated that methods and compositions employing topically applied functional inhibitors of acid sphingomyelinase (FIASMs) surprisingly ameliorate and prevent photosensitivity. A variety of compounds are known to be FIASMs, such as, for example, tricyclic antidepressants (TCAs), such as imipramine, and other related molecules, such as, for example, sertraline. As disclosed and exemplified herein, in some embodiments, TCAs can act as acid sphingomyelinase (aSMase) inhibitors for topical application for providing protection from or ameliorating of photosensitivity, particularly exposure to UV rays, either alone or combined with UV filters (e.g., sunscreens) or other topicals such as anti-rosacea medications (e.g., metronidazole, clindamycin). In some alternate embodiments, FIASMs, including TCAs, can be topically administered to augment the effectiveness and lessen the side effects of PDT. As exemplified herein, the disclosure demonstrates the pharmacologic effect of FIASMS, including but not limited to TCAs, to inhibit the enzyme aSMase involved in microvesicle particle (MVP) formation and release that is associated with photosensitivity.

In accordance with the results demonstrated herein, in various embodiments, the instant disclosure provides compositions, methods, regimens, systems, and articles of manufacture for providing protection from or ameliorating the damaging effects of UV radiation and PDT light sources, in particular in individuals with photosensitivity or in those being treated with topical photodynamic therapy.

In various embodiments, the methods and compositions for providing protection from or ameliorating of photosensitivity or improvement of topical photodynamic therapy include topically-applied FIASMs, for example, but not limited to, tricyclic antidepressants and other related compounds that act as functional inhibitors of aSMase, including, but not limited to, imipramine. In accordance with the invention, administration would not include oral or injected administration. As exemplified herein, topically applied imipramine is shown to inhibit the release of MVPs from skin cells, putatively by inhibition of a specific enzyme, aSMase, and in clinical studies imipramine is shown to reduce or relieve the physical effects of UV light exposure in otherwise photosensitive skin.

Topical application in a mouse model of photosensitivity associated with exaggerated MVP release in response to UVB has demonstrated reductions in MVP release and concomitant reductions in inflammatory cytokine production and erythema induced by UVB irradiation. Ex vivo human skin tissue experiments have demonstrated that topical application of the drug reduces markers associated with inflammation. Imipramine and its class analogs of tricyclic antidepressants and other related FIASMs have never before been used in a topical modality for relieving or preventing physical effects of photosensitivity, and indeed it has been historically believed that FIASMs, in particular TCAs, increase photosensitivity such that use of such compounds would be discouraged and expected to exacerbate conditions associated with photosensitivity.

In a first embodiment, the disclosure provides a skin protective composition for topical application to keratinous tissue comprising: a functional inhibitor of acid sphingomyelinase (FIASM); and a dermatologically acceptable carrier.

In some embodiments the FIASM is a compound selected from the group consisting of amineptine, amitriptyline, amitripylinoxide, amoxapine, butriptyline, clomipramine, demexiptiline, desipramine, dibenzepin, dimetacrine, dosulepin, doxepine, imipramine, imipraminoxide, iprindole, lofepramine, melitracen, metapramine, nitroxazepine, nortriptyline, noxiptiline, pipofezine, propizenpine, protriptyline, quinupramine, tianeptine, trimipramine, and combinations thereof.

In some embodiments the FIASM is present at a concentration in a range from about 0.1% to about 5% by weight based on the weight of the skin protective composition.

In some embodiments the carrier comprising one or a combination of ingredients selected from water, water-based solvents, oils, oil-based solvents, antimicrobials, antibiotics, redness reducers, antioxidants, emollients, and combinations thereof.

In some embodiments the carrier comprising at least one dermal penetrating enhancer selected from the group consisting of microemulsions, alcohols, polyols, surfactants, fatty acids, amines, amides, terpenes, sulfoxides, esters, and combinations of these.

In some embodiments when applied to skin, the skin protective composition demonstrates inhibition of acid sphingomyelinase, reduces or eliminates inflammation, reduces or eliminates microvesicle particle release, reduces or eliminates erythema, or a combination thereof.

In some embodiments the skin protective composition includes imipramine present in a range from about 2% to about 5% by weight based on the weight of the skin protective composition, and propylene glycol present in a range from about 1% to about 25% by weight based on the weight of the skin protective composition.

In some embodiments the skin protective composition further includes at least one additive selected from the group consisting of vitamins, fragrances, dyes, preservatives, oils, essential oils, salts, neutralizing or pH-adjusting agents, and combinations thereof.

In some embodiments the skin protective composition further includes at least one UV filter.

In some embodiments the at least one UV filter is selected from the group consisting of titanium dioxide; zinc oxide; iron oxides; cerium oxides; zirconium oxides; anthranilic compounds; dibenzoylmethane compounds; cinnamic compounds; salicylic compounds; camphor compounds; benzophenone compounds; diphenylacrylate compounds; triazine compounds; benzotriazole compounds; benzalmalonate compounds; benzimidazole compounds; imidazoline compounds; bis-benzoazolyl compounds; p-aminobenzoic acid (PABA) compounds;

methylenebis(hydroxyphenylbenzotriazole) compounds; benzoxazole compounds; dimers derived from α-alkylstyrene; 4,4-diarylbutadienes compounds; guaiazulene and derivatives thereof; rutin and derivatives thereof; flavonoids; bioflavonoids; oryzanol and derivatives thereof; quinic acid and derivatives thereof; and combinations thereof.

In some embodiments the skin protective composition is a cream, a gel, an ointment, or a spray, and is anhydrous, a water-in-oil emulsion, or an oil-in-water emulsion.

In another embodiment, the disclosure provides a method of ameliorating a skin condition in a subject, the method comprising: topically administering to a subject a skin protective composition comprising a functional inhibitor of acid sphingomyelinase (FIASM), the FIASM being present in a dermatologically acceptable carrier comprising one or a combination of ingredients selected from the group consisting of water, water-based solvents, oils, oil-based solvents, antimicrobials, antibiotics, redness reducers, antioxidants, emollients, and combinations thereof.

In some embodiments the topically administering ameliorates photosensitivity in the subject when the subject is exposed to a UV source of radiation.

In some embodiments the subject has been diagnosed with one or more of lupus erythematosus and rosacea prior to the administering step.

In some embodiments the subject is a human.

In some embodiments the FIASM is a compound selected from the group consisting of amineptine, amitriptyline, amitripylinoxide, amoxapine, butriptyline, clomipramine, demexiptiline, desipramine, dibenzepin, dimetacrine, dosulepin, doxepine, imipramine, imipraminoxide, iprindole, lofepramine, melitracen, metapramine, nitroxazepine, nortriptyline, noxiptiline, pipofezine, propizenpine, protriptyline, quinupramine, tianeptine, trimipramine, and combinations thereof.

In some embodiments the FIASM is present at a concentration in a range from about 0.1% to about 5% by weight based on the weight of the skin protective composition.

In some embodiments the skin protective composition includes imipramine present in a range from about 0.1% to about 5% by weight based on the weight of the skin protective composition, and propylene glycol present in a range from about 1% to about 25% by weight based on the weight of the skin protective composition.

In some embodiments the method further includes administering the skin protective composition at a frequency of at least once prior to exposure to sun, after exposure to sun, or both.

In some embodiments the skin protective composition is a cream, a gel, an ointment, or a spray, and is anhydrous, a water-in-oil emulsion, or an oil-in-water emulsion.

In another embodiment, the disclosure provides a skin care system for topical application to keratinous tissue comprising: a skin protective component comprising (i) a functional inhibitor of acid sphingomyelinase (FIASM); and (ii) a dermatologically acceptable carrier; and a photosensitizing component comprising at least one photosensitizer.

In some embodiments the FIASM is a compound selected from the group consisting of amineptine, amitriptyline, amitripylinoxide, amoxapine, butriptyline, clomipramine, demexiptiline, desipramine, dibenzepin, dimetacrine, dosulepin, doxepine, imipramine, imipraminoxide, iprindole, lofepramine, melitracen, metapramine, nitroxazepine, nortriptyline, noxiptiline, pipofezine, propizenpine, protriptyline, quinupramine, tianeptine, trimipramine, and combinations thereof.

In some embodiments the FIASM is present at a concentration in a range from about 0.1% to about 5% by weight based on the weight of the skin protective composition.

In some embodiments the carrier comprising one or a combination of ingredients selected from water, water-based solvents, oils, oil-based solvents, antimicrobials, antibiotics, redness reducers, antioxidants, emollients, and combinations thereof.

In some embodiments the carrier comprising at least one dermal penetrating enhancer selected from the group consisting of microemulsions, alcohols, polyols, surfactants, fatty acids, amines, amides, terpenes, sulfoxides, esters, and combinations of these.

In some embodiments when applied to skin, the skin care composition demonstrates inhibition of acid sphingomyelinase, reduces or eliminates inflammation, reduces or eliminates microvesicle particle release, reduces or eliminates erythema, or a combination thereof.

In some embodiments the skin protective composition includes imipramine present in a range from about 2% to about 5% by weight based on the weight of the skin protective composition, and propylene glycol present in a range from about 1% to about 25% by weight based on the weight of the skin protective composition.

In some embodiments the skin protective composition further includes at least one additive selected from the group consisting of vitamins, fragrances, dyes, preservatives, oils, essential oils, salts, neutralizing or pH-adjusting agents, and combinations thereof.

In some embodiments the skin protective composition further includes at least one UV filter.

In some embodiments the at least one UV filter is selected from the group consisting of titanium dioxide; zinc oxide; iron oxides; cerium oxides; zirconium oxides; anthranilic compounds; dibenzoylmethane compounds; cinnamic compounds; salicylic compounds; camphor compounds; benzophenone compounds; diphenylacrylate compounds; triazine compounds; benzotriazole compounds; benzalmalonate compounds; benzimidazole compounds; imidazoline compounds; bis-benzoazolyl compounds; p-aminobenzoic acid (PABA) compounds; methylenebis(hydroxyphenylbenzotriazole) compounds; benzoxazole compounds; dimers derived from α-alkylstyrene; 4,4-diarylbutadienes compounds; guaiazulene and derivatives thereof; rutin and derivatives thereof; flavonoids; bioflavonoids; oryzanol and derivatives thereof; quinic acid and derivatives thereof; and combinations thereof.

In some embodiments the skin protective composition is a cream, a gel, an ointment, or a spray, and is anhydrous, a water-in-oil emulsion, or an oil-in-water emulsion.

In some embodiments the at least one photosensitizer is selected from the group consisting of aminolevulinic acid, methyl aminolevulinate, 5-aminileuvulinic acid, porphyrin, protoporfin IX, purlytin, verteporfin, HPPH, temoporfin, methylene blue, photofrin, hematoporphyrin, Talaporfin, benzopophyrin derivative monoacid, Lutetium texaphyrin, metallophthalocyanine, metallo-naphthocyaninesulfobenzo-porphyrazine, metallo-naphthalocyanines, zinc tetrasulfophthalocyanine, bacteriochlorins, metallochlorins, chlorine derivative, Tetra(m-hydroxyphenyl)chlorin (mTHPC), pheophorbide, dibromofluorescein (DBF), IR700DX, naphthalocyanine, porphyrin derivative, and combinations thereof.

In some embodiments the photosensitizing component comprises a dermatologically acceptable carrier comprising one or a combination of ingredients selected from the group consisting of water, water-based solvents, oils, oil-based solvents, antimicrobials, antibiotics, redness reducers, antioxidants, emollients, and combinations thereof.

In another embodiment, the disclosure provides a method of reducing side effects resulting from a topical photodynamic therapy treatment, the method comprising: topically administering to a skin surface of a subject first and second compositions, simultaneously or sequentially, in any order, (i) the first composition comprising a functional inhibitor of acid sphingomyelinase (FIASM), the FIASM being present in a dermatologically acceptable carrier comprising one or a combination of ingredients selected from the group consisting of water, water-based solvents, oils, oil-based solvents, antimicrobials, antibiotics, redness reducers, antioxidants, emollients, and combinations thereof, and (ii) the second composition comprising at least one photosensitizer alone or present in in a dermatologically acceptable carrier comprising one or a combination of ingredients selected from the group consisting of water, water-based solvents, oils, oil-based solvents, antimicrobials, antibiotics, redness reducers, antioxidants, emollients, and combinations thereof; and applying a light source suitable for inducing the photosensitizer to at least the skin surface of the subject to which the first and second compositions are applied, wherein application of the first composition demonstrates inhibition of acid sphingomyelinase, reduces or eliminates inflammation, reduces or eliminates microvesicle particle release, reduces or eliminates erythema, or a combination thereof.

In some embodiments the topically administering the first and second compositions occurs prior to applying the light source suitable for inducing the photosensitizer.

In some embodiments the topically administering the first and second compositions occurs after applying the light source suitable for inducing the photosensitizer.

In some embodiments the topically administering the first and second compositions occurs while applying the light source suitable for inducing the photosensitizer.

In some embodiments comprising topically administering the first composition, followed by applying the light source suitable for inducing the photosensitizer, followed by topically administering the second composition.

In some embodiments comprising topically administering the second composition, followed by applying the light source suitable for inducing the photosensitizer, followed by topically administering the first composition.

In some embodiments the subject is a human.

In some embodiments the FIASM is a compound selected from the group consisting of amineptine, amitriptyline, amitripylinoxide, amoxapine, butriptyline, clomipramine, demexiptiline, desipramine, dibenzepin, dimetacrine, dosulepin, doxepine, imipramine, imipraminoxide, iprindole, lofepramine, melitracen, metapramine, nitroxazepine, nortriptyline, noxiptiline, pipofezine, propizenpine, protriptyline, quinupramine, tianeptine, trimipramine, and combinations thereof.

In some embodiments the FIASM is present at a concentration in a range from about 0.1% to about 5% by weight based on the weight of the skin protective composition.

In some embodiments the at least one photosensitizer is selected from the group consisting of aminolevulinic acid, methyl aminolevulinate, 5-aminileuvulinic acid, porphyrin, protoporfin IX, purlytin, verteporfin, HPPH, temoporfin, methylene blue, photofrin, hematoporphyrin, Talaporfin, benzopophyrin derivative monoacid, Lutetium texaphyrin, metallophthalocyanine, metallo-naphthocyaninesulfobenzo-porphyrazine, metallo-naphthalocyanines, zinc tetrasulfophthalocyanine, bacteriochlorins, metallochlorins, chlorine derivative, Tetra(m-hydroxyphenyl)chlorin (mTHPC), pheophorbide, dibromofluorescein (DBF), IR700DX, naphthalocyanine, porphyrin derivative, and combinations thereof.

In some embodiments each of the first and second composition is selected from a cream, a gel, an ointment, or a spray, and is anhydrous, a water-in-oil emulsion, or an oil-in-water emulsion.

In another embodiment, the disclosure provides an article of manufacture for a skin care system for topical application to keratinous tissue comprising at least first and second separately contained compositions, the first composition comprising a. skin protective component comprising (i) a functional inhibitor of acid sphingomyelinase (FIASM); and (ii) a dermatologically acceptable carrier, and the second composition comprising photosensitizing component comprising at least one photosensitizer, wherein the first and second compositions are contained to be separately dispensed for application to skin, in any order, and wherein the skin care system for topical application to keratinous tissue may be used according to any one of a number of possible embodiments of a skin care regimen, wherein the skin protective component is used in conjunction with the photosensitizing component, applied in sequence, each component applied once, or more frequently, in conjunction with delivery of photodynamic therapy employing a light source.

In another embodiment, the disclosure provides an article of manufacture for a skin care system for topical application to keratinous tissue comprising at least first and second separately contained compositions, the first composition comprising a skin protective component comprising (i) a functional inhibitor of acid sphingomyelinase (FIASM); and (ii) a dermatologically acceptable carrier, and the second composition comprising (i) a component selected from the group consisting of antimicrobials, antibiotics, redness reducers, antioxidants, UV filters, and combinations thereof; and (ii) a dermatologically acceptable carrier, wherein the first and second compositions are contained to be separately dispensed for application to skin, in any order, and wherein the skin care system for topical application to keratinous tissue may be used according to any one of a number of possible embodiments of a skin care regimen, wherein the skin protective component is used in conjunction with the at least one skin care active, applied in sequence, each component applied once, or more frequently, with applications of at least one of the components on a daily basis for a few or several days.

In another embodiment, the disclosure provides a regimen for enhancing skin, comprising: providing a first composition a skin protective component comprising (i) a functional inhibitor of acid sphingomyelinase (FIASM); and (ii) a dermatologically acceptable carrier comprising one or more ingredient selected from the group consisting of water, water-based solvents, oils, oil-based solvents, emollients, vitamins, fragrances, dyes, preservatives, oils, essential oils, salts, neutralizing or pH-adjusting agents, dermal penetrating enhancers, and combinations thereof, the carrier optionally comprising at least one selected from the group consisting of microemulsions, alcohols, polyols, surfactants, fatty acids, amines, amides, terpenes, sulfoxides, esters, and combinations thereof; and providing a second composition comprising (i) at least one skin care active selected from the group consisting of antimicrobials, antibiotics, redness reducers, antioxidants, UV filters, and combinations thereof; and (ii) a dermatologically acceptable carrier comprising one or more ingredient selected from the group consisting of water, water-based solvents, oils, oil-based solvents, emollients, vitamins, fragrances, dyes, preservatives, oils, essential oils, salts, neutralizing or pH-adjusting agents, dermal penetrating enhancers, and combinations thereof, the carrier optionally comprising at least one selected from the group consisting of microemulsions, alcohols, polyols, surfactants, fatty acids, amines, amides, terpenes, sulfoxides, esters, and combinations thereof, wherein the first and second compositions are contained to be separately dispensed for application to skin, in any order; applying the first composition to a region of skin of a subject; and applying the second composition to a region of skin of a subject, wherein the first and second compositions are applied sequentially, in any order.

In some embodiments the step of applying one or both of the first composition and the second composition is repeated daily for a period of time from one day to ten days, and wherein the regimen results in inhibition of acid sphingomyelinase, reduces or eliminates inflammation, reduces or eliminates microvesicle particle release, reduces or eliminates erythema, or a combination thereof.

In another embodiment, the disclosure provides a regimen for enhancing skin, comprising: providing at least first and second separately contained compositions, the first composition comprising a skin protective component comprising (i) a functional inhibitor of acid sphingomyelinase (FIASM); and (ii) a dermatologically acceptable carrier, and the second composition comprising a photosensitizing component comprising at least one photosensitizer, wherein the first and second compositions are contained to be separately dispensed for application to skin, in any order, and wherein the skin care system for topical application to keratinous tissue may be used according to any one of a number of possible embodiments of a skin care regimen, wherein the skin protective component is used in conjunction with the photosensitizing component, applied in sequence, each component applied once, or more frequently, in conjunction with delivery of photodynamic therapy employing a light source; providing at least a third composition a skin protective component comprising (i) a functional inhibitor of acid sphingomyelinase (FIASM); and (ii) a dermatologically acceptable carrier, wherein the third composition is contained to be separately dispensed for application to skin after the delivery of photodynamic therapy; and applying the at least a third composition to a region of skin of a subject to which the photodynamic therapy was delivered, wherein the at least a third composition is applied at least once per day over a period of at least three days following the delivery of photodynamic therapy.

In some embodiments the steps of applying one or both of the first composition and the third composition results in inhibition of acid sphingomyelinase, reduces or eliminates inflammation, reduces or eliminates microvesicle particle release, reduces or eliminates erythema, or a combination thereof.

In yet other embodiments, the disclosure provides a variety of other methods in compositions.

In some embodiments, the disclosure provides a method of ameliorating photosensitivity in a subject includes topically administering to a subject a skin protective composition including a therapeutically effective amount of a FIASM. The FIASM is present in a dermatologically acceptable carrier including one or a combination of ingredients selected from the group consisting of water, water-based solvents, oils, oil-based solvents, antimicrobials, antibiotics, redness reducers, antioxidants, emollients, and combinations thereof. The therapeutically effective amount is demonstrated to reduce or eliminate inflammation, microvesicle particle release, erythema, or a combination thereof. The FIASM demonstrates inhibition of acid sphingomyelinase.

In other embodiments, a skin protective composition includes a therapeutically effective concentration of a FIASM in a dermatologically acceptable carrier. The dermatologically acceptable carrier includes one or a combination of ingredients selected from the group consisting of water, water-based solvents, oils, oil-based solvents, antimicrobials, antibiotics, redness reducers, antioxidants, emollients, and combinations thereof. The FIASM demonstrates inhibition of acid sphingomyelinase.

In yet other embodiments, a method of inhibiting the release of microvesicle particles from skin cells includes topically applying to the skin of a subject a therapeutically effective amount of a FIASM. The FIASM is present in a dermatologically acceptable carrier including one or a combination of ingredients selected from the group consisting of water, water-based solvents, oils, oil-based solvents, antimicrobials, antibiotics, redness reducers, antioxidants, emollients, and combinations thereof. The FIASM demonstrates inhibition of acid sphingomyelinase.

In other embodiments, a method of reducing side effects resulting from a topical photodynamic therapy treatment includes topically administering to a subject a skin protective composition including a therapeutically effective amount of a FIASM. The FIASM is present in a dermatologically acceptable carrier including one or a combination of ingredients selected from the group consisting of water, water-based solvents, oils, oil-based solvents, antimicrobials, antibiotics, redness reducers, antioxidants, emollients, and combinations thereof. The therapeutically effective amount is demonstrated to reduce or eliminate an immunosuppressive effect from topical photodynamic therapy. The FIASM demonstrates inhibition of acid sphingomyelinase.

In yet other embodiments, a skin protective composition includes a therapeutically effective concentration of a FIASM in a dermatologically acceptable carrier. The dermatologically acceptable carrier includes one or a combination of ingredients selected from the group consisting of water, water-based solvents, oils, oil-based solvents, antimicrobials, antibiotics, redness reducers, antioxidants, emollients, and combinations thereof. The therapeutically effective concentration is demonstrated to reduce or eliminate an immunosuppressive effect from topical photodynamic therapy. The FIASM demonstrates inhibition of acid sphingomyelinase.

In yet other embodiments, a method of addressing a skin condition in a subject includes topically administering to a subject a skin care composition including an effective amount of a functional inhibitor of acid sphingomyelinase. The FIASM is present in a dermatologically acceptable carrier comprising one or a combination of ingredients selected from the group consisting of water, water-based solvents, oils, oil-based solvents, antimicrobials, antibiotics, redness reducers, antioxidants, emollients, and combinations thereof. The effective amount inhibits acid sphingomyelinase.

In yet other embodiments, a skin care composition includes an effective concentration of a functional inhibitor of acid sphingomyelinase in a dermatologically acceptable carrier. The dermatologically acceptable carrier includes one or a combination of ingredients selected from the group consisting of water, water-based solvents, oils, oil-based solvents, antimicrobials, antibiotics, redness reducers, antioxidants, emollients, and combinations thereof. The effective concentration demonstrates inhibition of acid sphingomyelinase.

In yet other embodiments, a method of inhibiting the release of microvesicle particles from skin cells includes topically applying to the skin of a subject an effective amount of a functional inhibitor of acid sphingomyelinase. The FIASM is present in a dermatologically acceptable carrier including one or a combination of ingredients selected from the group consisting of water, water-based solvents, oils, oil-based solvents, antimicrobials, antibiotics, redness reducers, antioxidants, emollients, and combinations thereof. The effective amount demonstrates inhibition of acid sphingomyelinase.

In yet other embodiments, a method of reducing an immunosuppressive effect in a subject undergoing a topical photodynamic therapy includes topically administering to a subject a skin care composition including an effective amount of a functional inhibitor of acid sphingomyelinase. The FIASM is present in a dermatologically acceptable carrier including one or a combination of ingredients selected from the group consisting of water, water-based solvents, oils, oil-based solvents, antimicrobials, antibiotics, redness reducers, antioxidants, emollients, and combinations thereof. The effective amount demonstrates inhibition of acid sphingomyelinase.

In yet other embodiments, a skin care composition includes an effective concentration of a functional inhibitor of acid sphingomyelinase in a dermatologically acceptable carrier. The dermatologically acceptable carrier includes one or a combination of ingredients selected from the group consisting of water, water-based solvents, oils, oil-based solvents, antimicrobials, antibiotics, redness reducers, antioxidants, emollients, and combinations thereof. The effective concentration is demonstrated to reduce or eliminate an immunosuppressive effect from topical photodynamic therapy.

### BRIEF DESCRIPTION OF THE DRAWINGS

FIG. 1 shows increased MVP release generated by UVB exposure in HaCaT Xeroderma Pigmentosum A (XPA)-deficient keratinocyte cell line in vitro compared to wild type (WT) HaCaT cells.

FIG. 2 shows increased MVP release generated in the skin by cutaneous UVB exposure of XPA knockout (KO) in comparison to wild-type (XPA-positive) mice in vivo, whereas MVP release in response to topical phorbol ester TPA and platelet-activating factor (PAF) agonist carbamoyl-PAF (CPAF) are similar amongst WT and XPA KO mice.

FIG. 3 shows increased MVP release generated by cutaneous UVB exposure in plasma of XPA KO in comparison to wild-type mice in vivo.

FIG. 4 shows inhibition of UVB-induced MVP release in vivo by topical imipramine in the skin tissue of wild-type and XPA KO mice.

FIG. 5 shows inhibition of UVB-induced MVP release in vivo by imipramine in the plasma of wild-type and XPA KO mice.

FIG. 6 shows that UVB-induced skin erythema responses (compared to baseline values) are much greater in XPA KO mice than wild-type counterparts.

FIG. 7 shows that treatment of mice with imipramine topically after UVB blocks the erythema responses, and although imipramine is effective in both wild-type and XPA KO mice, it is far more effective in the latter.

FIG. 8 shows that UVB-induced induction of the pro-inflammatory cytokine TNFα is upregulated to a greater extent in XPA KO mice over wild-type counterparts.

FIG. 9 shows that UVB-induced induction of the pro-inflammatory cytokine IL-6 is upregulated to a greater extent in XPA KO mice over wild-type counterparts.

FIG. 10 shows that post-UVB treatment with topical imipramine blocks TNFα mRNA responses in mice.

FIG. 11 shows that post-UVB treatment with topical imipramine blocks IL-6 mRNA responses in mice.

FIG. 12 shows that UVB-induced MVP generation, erythema responses, and IL-6/TNFα cytokine mRNA upregulation are exaggerated in the XPA KO mouse and blunted in the aSMase KO mouse.

FIG. 13 shows that both pretreatment of topical imipramine and post-treatment of imipramine following UVB blocks MVP generation in human skin explant tissue ex vivo.

FIG. 14 shows MVP release in HaCaT keratinocytes under varying conditions including PDT with various fluences of blue light, where the PDT consists of 5-ALA incubation followed by blue light treatment. Typically, in the clinic, 10-J blue light is employed for patient treatments.

FIG. 15 shows MVP release in human skin explants ex vivo under varying conditions including PDT.

FIG. 16 shows inhibition of MVP generation by HaCaT keratinocytes in vitro by imipramine following exposure to PDT.

FIG. 17 shows inhibition by topical imipramine of MVP generation by human skin tissue explants ex vivo following exposure to PDT, where topical responses to phorbol ester TPA and PAF agonist CPAF are included as positive controls.

FIG. 18 shows inhibition by topical imipramine of MVP generation following PDT in vivo in a mouse model employing wild-type mice.

FIG. 19 shows inhibition of MVP generation in the absence of aSMase enzyme (i.e., aSMase KO mice) in a mouse model in response to topical PDT, which confirms that MVP release in response to PDT requires aSMase.

FIG. 20 shows results of contact hypersensitivity protocols measuring skin reactions to the neoantigen dinitrofluorobenzene in wild-type mice, which indicates that topical imipramine following PDT blocks PDT-induced local and systemic immunosuppression but has no effect on the systemic immunosuppression induced by systemic exposure to the PAF receptor (PAFR) agonist CPAF.

FIG. 21 shows that topical PDT treatment of aSMase-deficient mice did not result in decreased systemic and local dinitrofluorobenzene (DNFB) responses, whereas these mice responded to intraperitoneal CPAF.

FIG. 22A shows that localized UVB irradiation of human subjects with photosensitivity results in increased MVP release over control subjects.

FIG. 22B shows inhibition by topical imipramine added following 1 kJ/m² UVB irradiation blocked the increased MVP release from localized UVB irradiation of forearm skin of human subjects with photosensitivity.

FIG. 23A shows that UVR treatment of XPA KO mice results in increased MVP release in skin dependent upon the PAFR.

FIG. 23B shows that UVR treatment of XPA KO mice results in increased MVP release in blood plasma dependent upon the PAFR.

FIG. 24A shows that application of topical 4% imipramine before UVB blocks the erythema response of low-fluence (300 J/m²) UVB on subjects with rosacea.

FIG. 24B shows photographic and thermal imaging (inset) results for one representative subject from FIG. 24A.

### DETAILED DESCRIPTION OF THE DISCLOSURE

Disclosed herein are methods and compositions formulated to avoid or ameliorate photosensitivity.

Also disclosed herein are methods and compositions formulated to enhance the efficacy of and avoid or ameliorate side effects of topical photodynamic therapy.

Methods and compositions for providing protection from or ameliorating for photosensitivity or for improved topical photodynamic therapy are provided using topically applied FIASMs, such as, but not limited to, imipramine. The imipramine is shown to inhibit the release of microvesicle particles from skin cells, putatively by inhibition of a specific enzyme, aSMase. Topical application in a mouse model demonstrates reductions in MVP release in response to UVB radiation or experimental PDT. Surprisingly, topical application blocks not only exaggerated MVP associated with XPA KO model of photosensitivity, but it also blocks the exaggerated erythema and inflammatory cytokine responses. Similarly, topical application of imipramine following experimental PDT blocks MVP release, as well as the immunosuppressive effects of this FDA-approved treatment modality. Ex vivo human skin tissue experiments demonstrate that topical application of the drug reduces MVP release in response to UVB or PDT. Imipramine and its class analogs of tricyclic antidepressants have never before been used in this specific formulation, disease, or treatment context and provide an option for photosensitivity drugs, prophylactics, and improved topical photodynamic therapy.

Where the following terms are used in this specification, they are used as defined below.

As used herein, the term “functional inhibitor of acid sphingomyelinase” or “FIASM” means and refers to compounds characterized as having a biological mechanism of action of inducing the detachment of aSMase enzyme from inner lysosomal membranes with its subsequent inactivation. Hence, these agents are functional inhibitors of the aSMase enzyme. In some embodiments, FIASMs are cationic amphipathic substances.

As used herein, the terms “optional” or “optionally” mean that the subsequently described event or circumstance may or may not occur and that the description includes instances where said event or circumstance occurs and instances where it does not.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, the subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been diagnosed with a need for treatment for cancer, such as, for example, prior to the administering step.

As used herein, the terms “treating” and

prophylaxis and treatment” refer to partially or completely alleviating, ameliorating, relieving, delaying onset of, inhibiting or slowing progression of, reducing severity of, and/or reducing incidence of one or more symptoms or features of a particular disease, disorder, and/or condition. Compositions can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be cancer.

As used herein, the terms “inhibit” or “inhibiting” and “reduce” or “reducing” and “ameliorate” mean decreasing or eliminating photosensitivity, as demonstrated by reducing, eliminating or avoiding one or more of inflammation, microvesicle particle release, and erythema, particularly in subjects who are known or expected to develop inflammation and/or redness or erythema due to a medical condition or treatment.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, less than 0.01 wt. %, or none of the specified material.

In some embodiments, a skin protective composition according to the disclosure includes a therapeutically effective concentration of a FIASM in a dermatologically acceptable carrier.

In some embodiments, the skin protective composition is topically administered to a subject.

In some embodiments, the therapeutically effective amount is demonstrated to reduce or eliminate inflammation, MVP release, and/or erythema.

Without wishing to be bound by theory, it is believed that, based on the Examples shown herein, a FIASM, when applied topically in a skin protective composition, reduce sun sensitivity and improve topical photodynamic therapy by inhibiting aSMase to reduce MVP production in keratinocytes, where the production of MVPs upon exposure to UV radiation is an underlying feature of sun sensitivity and the negative side effects associated with topical photodynamic therapy. The ability of topically-applied FIASM to ameliorate UV exposure and side effects of topical photodynamic therapy is surprising and unexpected, because many FIASMs, when administered orally, have been understood to increase photosensitivity leading to warnings about sun and other sources of UV exposure during their use.

### Ameliorating Sun and UV Sensitivity

In some embodiments, a topical administration of a skin protective composition according to the disclosure ameliorates photosensitivity in the subject.

In some embodiments, the subject has been previously diagnosed with one or more medical conditions associated with photosensitivity, which may include, but are not limited to, lupus erythematosus and/or rosacea.

Xeroderma Pigmentosum is a family of genetic disorders in which the ability to repair UVB-damaged DNA is decreased. The XPA protein is known to recognize UVB photoproducts in DNA and facilitate damage removal by nucleotide excision repair. XPA deficiency therefore decreases UVB photoproduct repair efficiency, is linked to photosensitivity, and therefore serves as a good model system for photosensitivity. Hence, tests were designed to test if XPA deficiency results in augmented UVB-induced MVPs. These MPVs are small pieces of the cells themselves, formed and released by blebbing from the cell membrane.

Tests of a spontaneously transformed aneuploid immortal keratinocyte-derived cell line from adult human skin (HaCaT), in which XPA was knocked down, revealed increased MVP release in response to UVB, the results of which are shown in Example 1. These in vitro results indicate that microvesicle generation was increased significantly in HaCaT XPA deficient cells compared to control HaCaT cells.

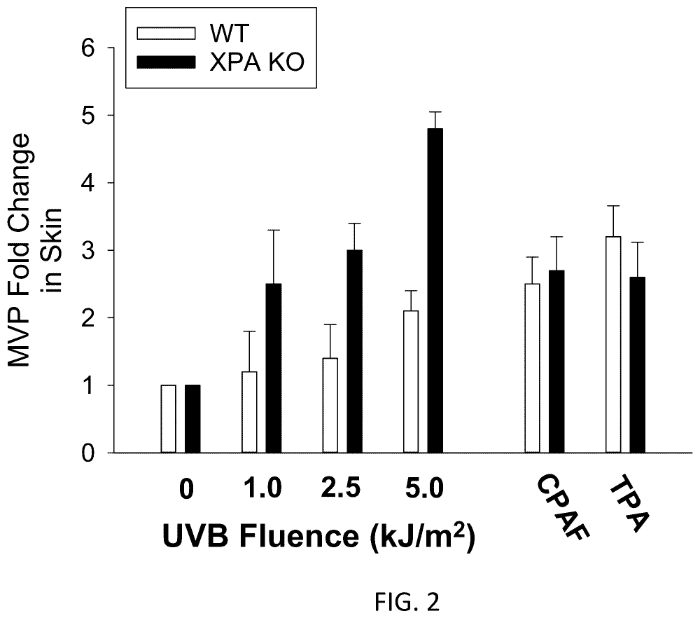

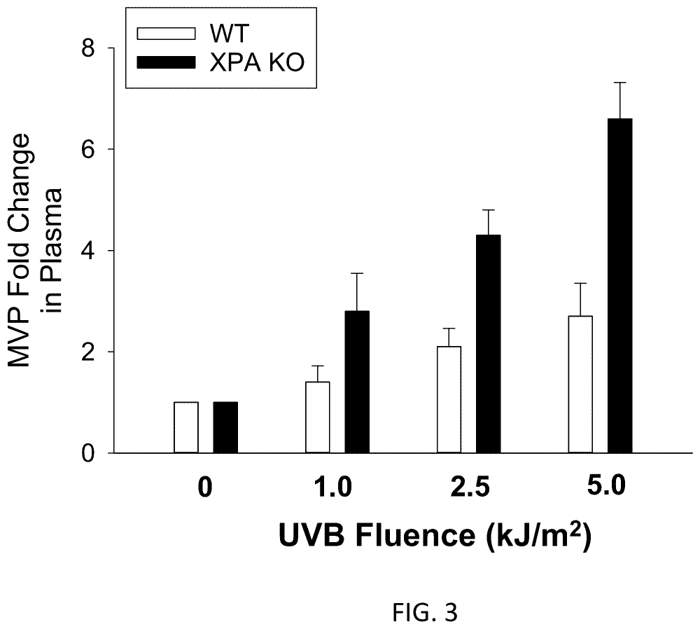

Similarly, as shown in Example 2, XPA-deficient mice generated increased MVPs in both skin tissue (see FIG. 2) and plasma (see FIG. 3) in response to UVB as compared to wild-type counterparts. However, the absence of XPA did not affect MVP release in response to PAFR agonist CPAF or phorbol ester TPA (see FIG. 2). As MVPs have been implicated in UVB signaling, these results suggest that these subcellular bodies could play a role in the augmented UVB responses associated with XPA-deficiency. As will be detailed herein, the experimental results with both pharmacologic and genetic approaches support the surprising discovery that MVP are involved in the increased inflammation associated with UVB responses.

The lipid mediator platelet-activating factor (PAF) is understood to play an important role in the augmented acute inflammation associated with UVB exposure in XPA-deficient mice. Indeed, it has previously reported that XPA-deficient cells and mice generate more PAF agonists in response to UVB, many produced non-enzymatically by oxidation of glycerophosphocholines in response to this pro-oxidative stressor. Moreover, the ability of UVB to generate subcellular MVPs is understood to be due to PAFR-mediated translocation and activation of the enzyme acid sphingomyelinase (aSMase).

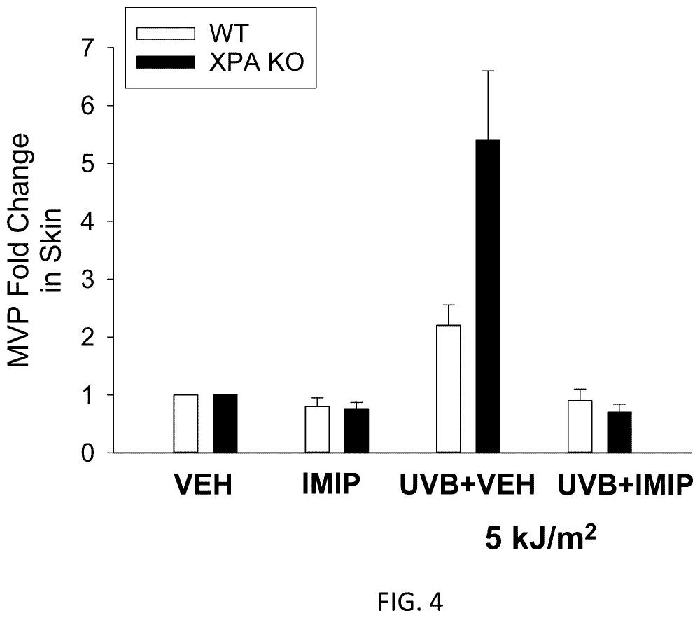

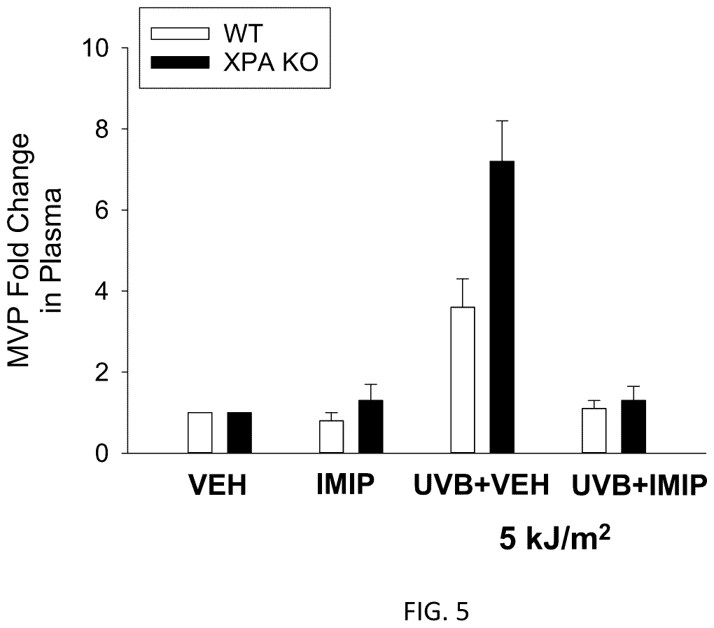

The enzyme aSMase is also known to generate MVP in response to many stressors such as UVB via activation of PAFR. Yet other stimuli such as in response to a phorbol ester (TPA) are independent of the PAFR. Therefore, administration of inhibitors of aSMase may block UVB-MVP release in XPA deficient model, as well as in other situations where MVP release is independent of the PAFR. Imipramine, a known aSMase inhibitor, as well as other FIASMs, may have an inhibitory effect on MVP generation. Anesthetized wild-type (WT) and XPA-deficient mice on C57/BL6 backgrounds were irradiated with 5 kJ/m² UVB and immediately after UVB treatment, to see whether Imipramine can block UVB-MVP release in XPA-deficient murine model, 100 ul of 500 μM imipramine was applied topically on the shaved back of mice. To determine the inhibition of MVP release by Imipramine, mice were also treated with UVB fluences followed by topical vehicle without imipramine. To confirm imipramine alone is not causing MVP generation, an imipramine alone mice group was also considered. Lastly, control group was considered that does not received UVB or imipramine but vehicle alone. Topical imipramine given post-UVB treatment blocked MVP accumulation in skin (see FIG. 4) and in plasma (see FIG. 5) in both WT and XPA KO mice.

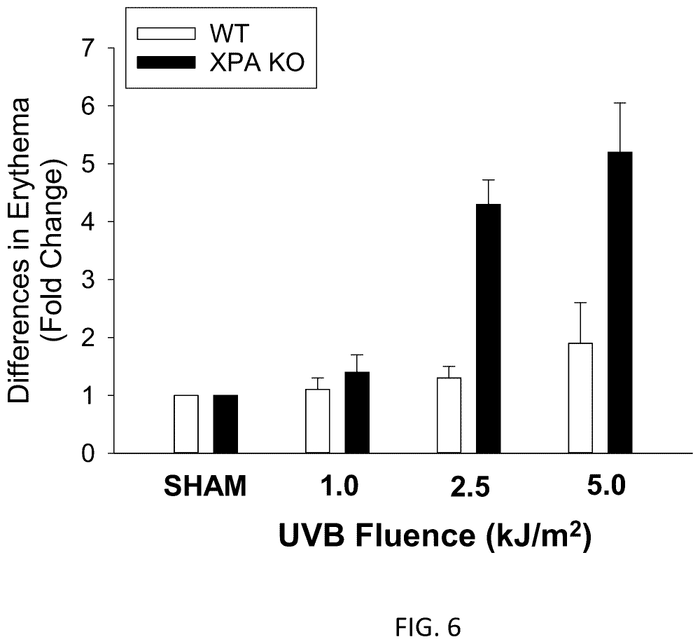

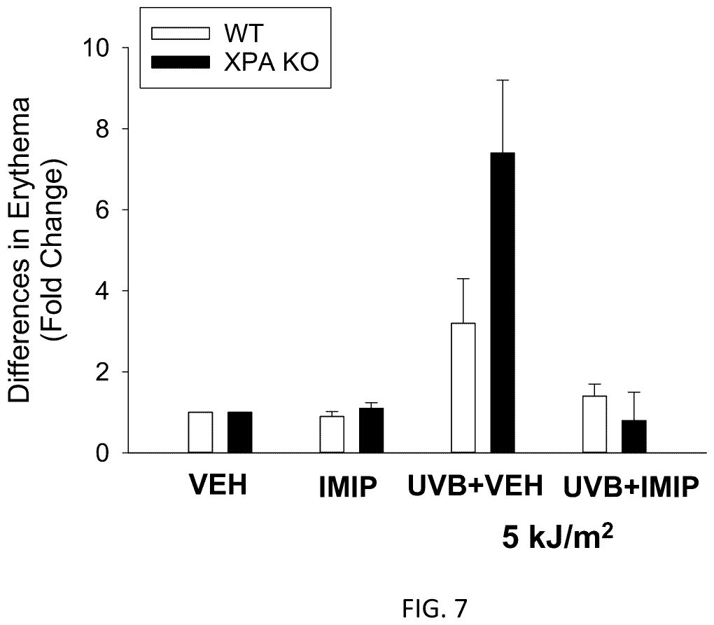

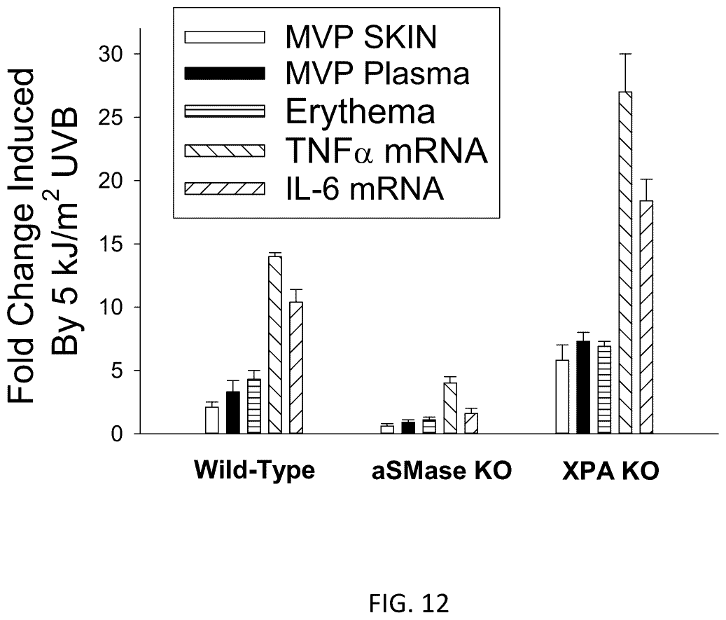

As erythema is an acute reaction to UVB, it was determined whether XPA KO mice exhibited enhanced acute erythema responses in response to various UVB fluences in comparison to WT mice. Erythema was measured by a Mexameter® device (Courage+Khazaka electronic GmbH, Cologne, Germany) before treatment and after 4 hours of treatment. UVB treatment alone showed greater erythema in the XPA KO in comparison to WT mice (see FIG. 6). Surprisingly, treatment with imipramine post-UVB blocked the erythema responses with the effectiveness of this agent most noted in the XPA KO mice (see FIG. 7). UVB-induced skin inflammation involves many cytokines including interleukin-6 (IL-6) and tumor necrosis factor-alpha (TNFα). As shown in FIG. 6 and FIG. 7, quantitative reverse transcription polymerase chain reaction (RT-qPCR) on RNA isolated from skin biopsies taken 4 h post UVB treatment resulted in the upregulation of mRNA levels for both of these cytokines, with greater responses noted in the XPA KO mice. Consistent with the erythema responses, topical imipramine given post-UVB irradiation inhibited the increased cytokine expression levels. Consistent with the pharmacologic approach using topical imipramine, UVB irradiation of mice with genetic KO of aSMase (Smpd1−/−) resulted in similar findings (see FIG. 12). It was previously determined that UVB does not generate MVP in aSMase KO mice.

Together, these data indicate that aSMase is required for UVB-induced MVP release and that MVP are responsible for acute inflammatory responses from UVB. Thus, the presented results provide significant premise for the surprising concept that blocking MVP release via targeting aSMase effectively via FIASM application blocks the erythema and cytokine responses associated with acute UVB exposure. It should be noted that although aSMase inhibition was effective in both WT and XPA KO mice, the effects were more dramatic in the latter due to the increased levels of these inflammatory endpoints. Therefore, other inhibitors of aSMase are expected to have a similar effect against photosensitivity as imipramine.

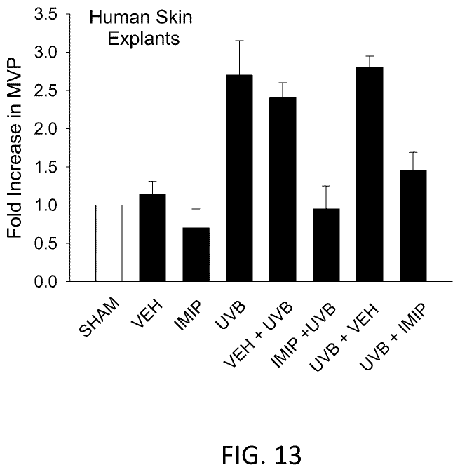

The ability of topical imipramine to block UVB-generated MVP release was determined ex vivo in human skin using discarded human skin explants from abdominoplasty surgeries. As shown in FIG. 13, topical imipramine, given either 1 h prior to or immediately following UVB irradiation, blocked MVP release. These results confirm that topical imipramine and presumably other FIASMs could be effective given either prior to or immediately following UVB exposure in human skin.

The surprising observation that topical imipramine application induces effects on MVP release and erythema responses in a preclinical model of photosensitivity establishes this drug and its analogs as a viable option for prophylaxis and active disease state treatment of conditions that involve photosensitivity, including, but not limited to, lupus erythematosus and rosacea. The methods and compositions according to the disclosure are thus suitable for generally addressing photosensitivity, and particularly in those subjects who are vulnerable to or suffer from photosensitivity as an aspect of a known dermatologic and/or systemic condition, or as a result of other factors, including dietary intake and drug included photosensitivity.

Of importance, Example 9 links the abnormal erythema and inflammatory responses associated with photosensitivity and ASM inhibition. In particular, Example 10 shows that topical treatment with an ASM inhibitor (functional inhibitor of ASM [FIASM]) results in decreased MVP release as well as decreased erythema and inflammatory responses in mice with photosensitivity. Further, Example 11 surprisingly indicates that pretreatment with topical ASM inhibitor imipramine can block the erythema reaction in response to a small amount of artificial UVB on the face of patients with the photosensitive disorder rosacea.

As noted herein, UV light acting on the skin keratinocytes results in increased MVP formation via activation of aSMase. Moreover, intermediate steps involve UV light causing the production of the lipid mediator PAF, which, by acting on the keratinocyte PAFR, stimulates the translocation of aSMase to the plasma membrane of keratinocytes, where it triggers the generation and release of MVP, which are bioactive. In photosensitive conditions, UV triggers more PAF, which then results in more MVP release, which helps mediate the acute excess inflammation associated with UV in patients who are photosensitive. As inhibitors of the PAF system are not available, TCAs, through their ability to block aSMase, a key factor in the MVP release pathway, appear to be a beneficial dermatological and pharmacologic treatment for these disorders.

### Topical Photodynamic Therapy

Topical PDT for “field therapy” of areas of actinic damage consists of treatment of the skin first with a topical sensitizer (usually 5-aminolevulinic acid [5-ALA]) followed by exposure to a blue light source for approximately 16 minutes. The results that garnered FDA approval for 5-ALA PDT used an overnight incubation of 5-ALA. However, because these conditions resulted in severe skin reactions, current protocols employ one or two hour incubations of 5-ALA prior to blue light exposure. Even under these modified conditions, the vast majority of patients receiving topical PDT find it very painful, which has limited its clinical use.

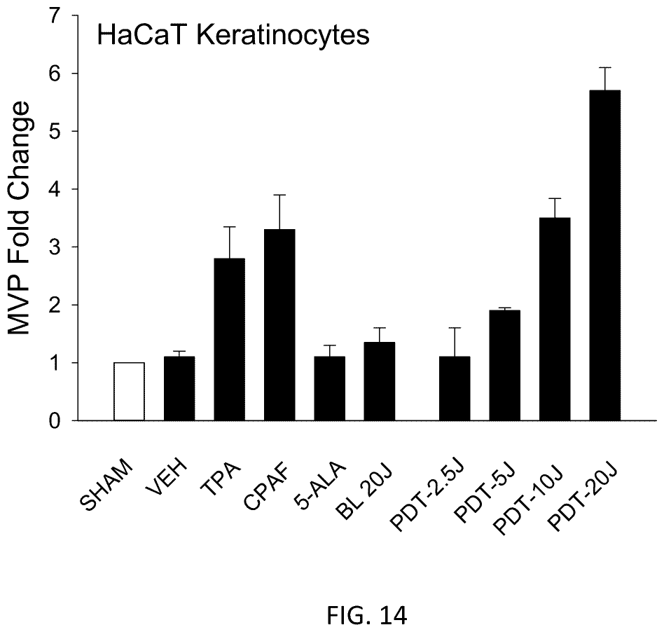

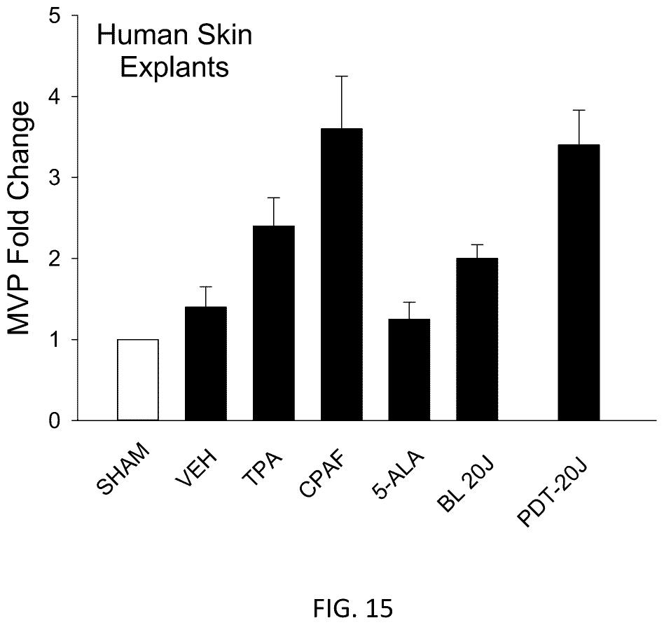

PDT results in acute cellular destruction and inflammation and photosensitivity, as well as delayed immunosuppression. Given that PDT has some commonality to UVB, it was of interest to demonstrate whether PDT resulted in MVP release, and, whether MVP release could be involved in the adverse acute and delayed effects of this modality. It was previously demonstrated that PDT resulted in high levels of PAF agonists HaCaT keratinocytes. Thus, it was of further interest whether experimental PDT of HaCaT keratinocytes generated MVP in vitro. To that end, HaCaT cells were pretreated with 5-ALA alone, or blue light alone, or a 4 hour incubation of 5-ALA in the dark followed by various fluences of blue light, and MVP were measured in the supernatants at 4 hours post blue light. 100 nM CPAF and TPA were used as positive controls for MVP release. As shown in FIG. 14, 5-ALA and 20 J blue light alone did not generate appreciate MVP formation in HaCaT keratinocyte cells. However, the combination of 5-ALA along with blue light (i.e., PDT) resulted in high levels of MVP generation in a dosage-dependent manner. Similarly, ex vivo results revealed that experimental PDT resulted in MVP release in human explant tissue (see FIG. 15).

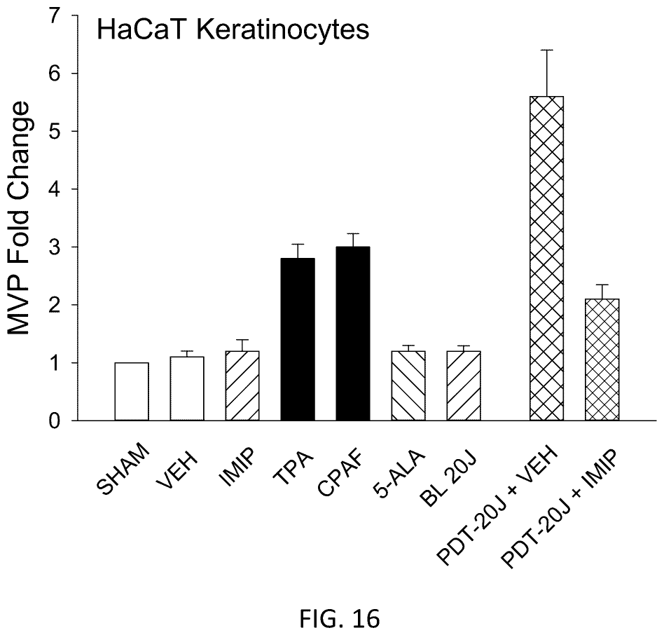

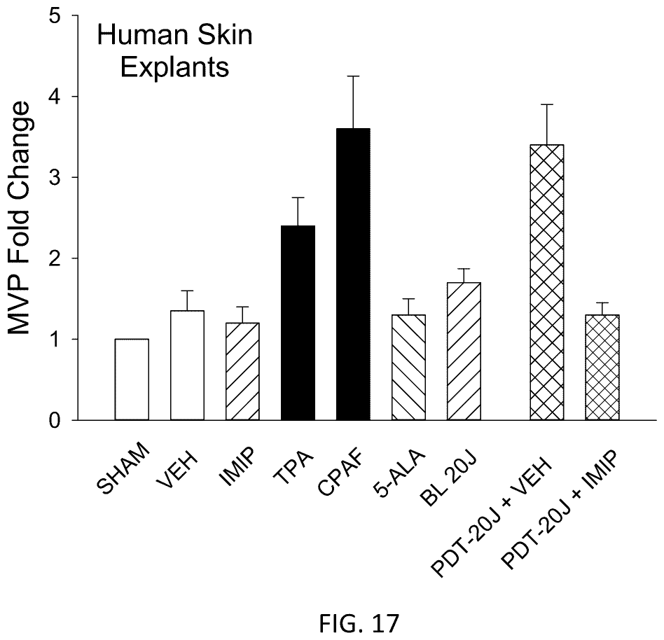

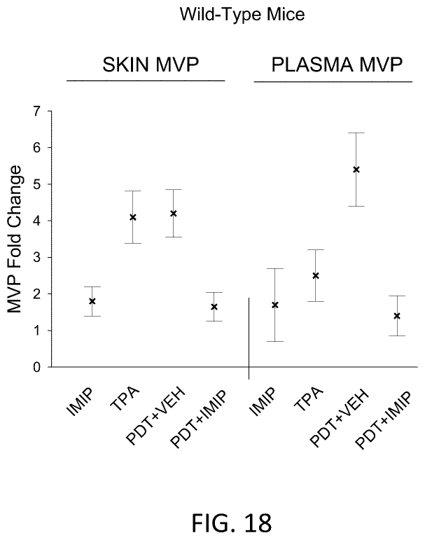

Next, the role of PAF and aSMase in PDT-induced MVP release was assessed. Though PDT is a potent generator of PAF, use of PAFR antagonists and cells and mice with/without PAFRs revealed that the PAFR was not necessary for PDT-induced MVP production (data not shown). However, surprisingly, in vitro results using HaCaT keratinocytes (see FIG. 16) and ex vivo results using human skin explants (see FIG. 17) both revealed that imipramine given post blue light exposure blocked PDT-induced MVP release. Next, the ability of topical PDT to generate MVP in vivo in mice, and the role of aSMase in this process, was determined. Wild-type mice underwent experimental PDT alone or PDT followed by topical application of imipramine following the 20-J blue light exposure. As shown in FIG. 18, PDT resulted in increased MVP release in both skin and plasma of mice. Topical TPA also resulted in increased levels of MVP. Two approaches were used to determine if PDT-induced MVP involved aSMase. Importantly, imipramine administration blocked MVP release. Use of aSMase KO mice mirrored the pharmacologic effects of topical imipramine, with aSMase KO mice not responding to PDT with MVP production. Of interest, topical application of the aSMase product C-2 ceramide generates MVP in the aSMase KO mice demonstrating that these mice can still generate MVP under some conditions, just not those stimuli dependent upon aSMase activation (data not shown).

Inasmuch as multiple publications have demonstrated that PDT is immunosuppressive, and this effect inhibits its effectiveness, it was of interest to determine that blocking MVP release via targeting aSMase could prevent its immunosuppressive effects. Of importance, in humans and mice, PDT has been demonstrated to be locally immunosuppressive, denoting that the area treated with PDT does not respond to either the sensitization or elicitation phases of contact hypersensitivity (CHS) reactions. In murine models PDT is also systemically immunosuppressive, which indicates that following PDT of an area, sensitization and elicitation reactions of other non-PDT-treated skin are blunted. Local immunosuppression is thought to involve compromised skin dendritic cell function. Systemic immunosuppression involves PAF and other agents produced by the keratinocyte which act upon the dermal mast cell, causing these cells to travel to local lymph nodes where immunosuppressive regulatory T cells are generated.

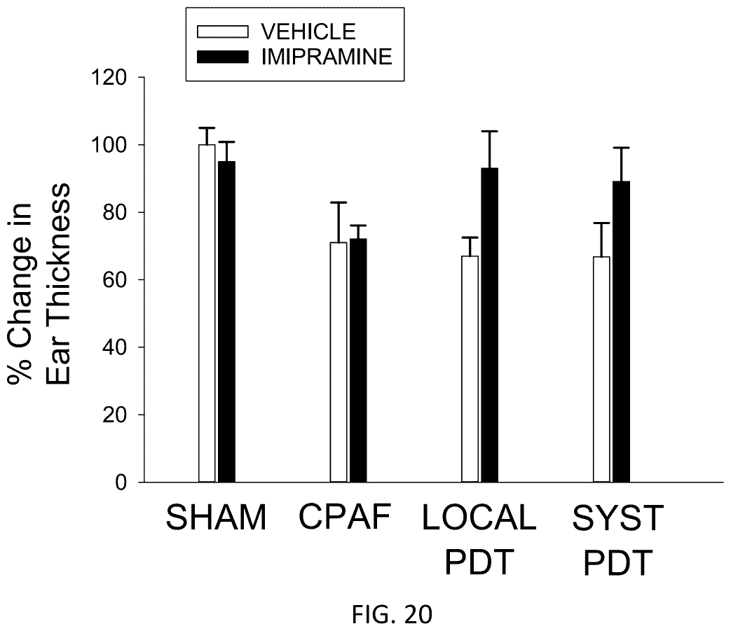

The ability of topical imipramine to block PDT-induced immunosuppression was determined in vivo using wild-type mice. PDT using 10-J was performed to an area of back skin, followed by treatment with topical imipramine or vehicle. Five days later the mice underwent sensitization with the neoantigen DNFB. To test for local immunosuppression, DNFB was applied to the PDT-treated area. To test for systemic immunosuppression, the DNFB was applied to back skin at least 2 cm away from the PDT-treated area. Nine days later the mice underwent elicitation reactions to DNFB by application to one ear, with vehicle applied to the other ear. One day later the ear thickness was obtained as a measure of CHS reaction and compared to pre-treatment and vehicle-treated values. Some mice were treated with intraperitoneal CPAF as a positive control for systemic immunosuppression. As shown in FIG. 20, PDT induced both local and systemic immunosuppressive responses as denoted by decreased CHS reactions to the neoantigen DNFB. Surprisingly, topical imipramine given post-PDT inhibited both local and systemic immunosuppression. However, topical imipramine did not affect the immunosuppressive effects of CPAF.

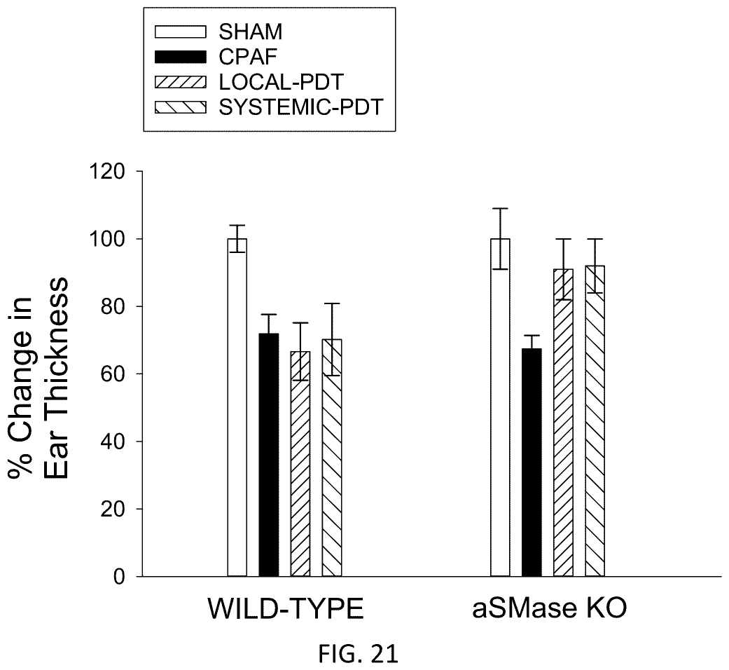

Next, a genetic approach of aSMase KO mice was used to assess their immunosuppressive responses to PDT. Shown in FIG. 21, mice lacking aSMase did not respond to PDT with local nor systemic immunosuppressive responses. However, treatment of these mice with the PAFR agonist CPAF resulted in similar levels of immunosuppression in comparison to wild-type mice.

These results demonstrate that PDT generates MVP, and these subcellular bodies appear to mediate the known immunosuppressive effects of this FDA-approved modality. Given that this bystander effect of immunosuppression from topical PDT could negatively impact the therapeutic efficacy of this modality commonly used field therapy for actinic damage, the present results suggest that agents that target aSMase, such as imipramine, may improve PDT effectiveness by reducing this immunosuppression effect.

According to the inventors' findings, TCAs appear to be a pharmacological treatment for improved topical photodynamic therapy through their ability to block aSMase, which also reduces MVP release and the immunosuppressive effects caused by topical PDT. Administration of FIASMs to a patient undergoing PDT may improve PDT effectiveness by reducing this immunosuppression effect. As topical TCAs such as amitriptyline and imipramine can also be used as nociceptives, it would be expected that their use along with PDT could improve the tolerability of this treatment.

### Product Forms

It will be appreciated that in the various embodiments, inventive compositions may be provided in any of a variety of product forms that include carrier and other active ingredients that are well known in the dermatologic, cosmetics, cosmeceuticals and topical drug delivery fields. Selection of carrier ingredients suitable for inclusion with the one or more functional acid sphingomyelinase inhibitors are based on the product form vehicles used which may be selected based on solubility and other features of a selected FIASM. The most common vehicles in dermatology are creams, ointments, gels, lotions, sprays and foams, which commonly employ one more of preservatives, stabilizing ingredients, pH buffers and related components of the respective vehicles.

In some embodiments, dermatologically acceptable carriers for creams may include one or more ingredients selected from the group consisting of propylene glycol, stearyl and cetyl alcohols, aluminum acetate basic, sorbitan monostearate, sodium lauryl sulfate, polysorbate 60, isopropyl myristate, sodium sulfite anhydrous, polysorbate 80, purified water, glycerin, mineral oil, white petrolatum, white wax, and combinations thereof.

In some embodiments, dermatologically acceptable carriers for ointments may include one or more ingredients selected from the group consisting of mineral oil, paraffin, propylene carbonate, white petrolatum, white wax, citric acid, 1,2,6-hexanetriol, polyethylene glycol, propylene glycol, stearyl alcohol, and combinations thereof.

In some embodiments, dermatologically acceptable carriers for lotions may include one or more ingredients selected from the group consisting of sodium lauryl sulfate, light liquid paraffin, cetyl alcohol, stearyl alcohol, propylene glycol, methyl hydroxybenzoate, propyl hydroxybenzoate, sorbitan monostearate, self-emulsifying glyceryl monostearate, edetate sodium, purified water, citric acid, sodium hydroxide, and combinations thereof.

In some embodiments, dermatologically acceptable carriers for gels may include one or more ingredients selected from the group consisting of carbomer homopolymer Type C, cocoyl caprylocaprate, fragrance, mineral oil, propylene glycol, strong ammonia solution, stearic acid, isopropyl myristate, polyoxyl 40 stearate, stearyl alcohol, xanthan gum, sorbic acid, butylated hydroxytoluene, purified water, and combinations thereof.

In some embodiments, dermatologically acceptable carriers for sprays may include one or more ingredients selected from the group consisting of alcohol, isopropyl myristate, sodium lauryl sulfate, glyceryl oleate, isopropyl alcohol, isopropyl myristate, L-menthol, mineral oil, undecylenic acid, and combinations thereof.

The above described product forms and the ingredients listed are non-limiting examples that are representative of product forms for providing the inventive compositions hereof. Additional representative embodiments of inventive composition ingredients are provided below.

### Compositions

In accordance with the various embodiments, the skin protective composition s hereof include one or a combination of ingredients. In some particular embodiments, the invention includes a skin protective composition comprising a functional inhibitor of acid sphingomyelinase (FIASM), which may further include a dermatologically acceptable carrier and optionally one or more additional ingredients.

In some embodiments, compositions may be aqueous, glycol based, alcohol based, or anhydrous. In some embodiments, a skin protective composition is formed by providing a dermatologically acceptable carrier having a water-phase that comprises water, wherein the dermatologically acceptable carrier may further comprise one more additional ingredients, each present in the water phase or in another phase, such as an oily phase. In some embodiments, a skin protective composition is formed by providing a dermatologically acceptable carrier having at least one oily-phase that excludes or is substantially free of water, wherein the dermatologically acceptable carrier may further comprise one more additional ingredients, each present in the at least one oily phase or in another phase. In some embodiments, the skin protective composition may include one or more of any one of additional ingredients. In some embodiments, the one or more additional ingredients includes one or more of a water, glycol, a humectant, a preservative, a surfactant, a thickener, an active compound, water-based solvents, oils, oil-based solvents, antimicrobials, antibiotics, redness reducers, antioxidants, emollients, emulsifiers, fragrances, pH adjusters, other dermatologically acceptable additives, or a combination thereof.

The skin protective composition may be any suitable form, such as, but not limited to, a single phase solution comprising a water phase, a multi-phase solution comprising at least one water phase, a single or multi-phase solution comprising at least one oily phase, an emulsion comprising a water phase, such as, but not limited to, a water-in-oil emulsion, an oil-in-water emulsion, or a silicone-in-water emulsion. The skin protective composition may be provided in a product form for application to keratinous tissue, such as, but not limited to, an applicator that includes one or more compositions according to the disclosure, and may be a product form such as, but not limited to, a cream, ointment, gel, lotion, spray, foam, hydroglycolic solution, or micellar water. The skin protective composition in any of the product forms may be either a leave-on or a rinse-off formulation.

In some particular embodiments, the invention includes systems that include two or more compositions, for example a skin protective component comprising a functional inhibitor of acid sphingomyelinase, and a photosensitizing component that comprises at least a photosensitizer, each of which components may further include a dermatologically acceptable carrier and optionally one or more additional ingredients. In accordance with the various embodiments, the compositions according to the disclosure include ingredients that are present in amounts by weight, typically based on the weight of the skin protective composition, wherein the ingredients may be present at a concentration, by weight, of from about 0.001% to about 100%. Thus, in various embodiments, an ingredient may be present in a composition in a weight percent amount from about 0.001, to about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 to about 99 percent by weight, or any suitable combination, sub-combination, range, or sub-range thereof by weight, based on the total weight of the skin protective composition. One of ordinary skill in the art, however, will appreciate that other ranges are within the scope of the invention.

### Functional Inhibitors of Acid Sphingomyelinase

In various embodiments, the methods and compositions according to the disclosure include use of FIASMs for providing protection from or ameliorating of photosensitivity, particularly photosensitivity induced by exposure to UV rays, or for improved topical photodynamic therapy.

In various embodiments, the FIASM is a compound that demonstrates inhibition of aSMase.

In some embodiments, the FIASM is a tricyclic antidepressant (TCA). According to the disclosure, tricyclic antidepressants, for example imipramine, such as, for example, commercially available under the trademark Tofranil® (SPECGX LLC, Webster Groves, MO), and that are known to inhibit the enzyme aSMase, are employed in the skin protective composition.

Imipramine is a generic drug used orally to treat depression, as well as pain syndromes. It is well-known that this class of medications, tricyclic antidepressants, can inhibit aSMase, a feature not necessarily involved in therapeutic effects for depression/pain. Imipramine has also been reported in the patent literature as an injectable for cancer treatment (see, for example, US Pat. App. Pub. No. 2021/0121475), and there have been reports of imipramine compounded with other agents used to treat pain syndromes. Notably, imipramine and other drugs in its class have long been considered to induce photosensitivity and patients receiving such drugs, in particular those taking the drugs by an oral route of administration, have been conspicuously advised to avoid exposure to the sun. Thus, it is indeed unexpected and not consistent with known properties that when applied topically and for the purpose of ameliorating photosensitivity that the benefits reported here can be realized.

According to the general literature, topical use of tricyclic antidepressants has not been associated with any side effects. Of note, there have been several reports of imipramine compounded with clonidine that resulted in toxic effects of clonidine, but imipramine levels were low/undetectable. Similarly, literature has reported that propylene glycol enhances absorption of tricyclic antidepressants (desipramine, nortriptyline, amitriptyline, imipramine) in hairless mice.

In some embodiments, treatments and compositions may include a FIASM. In some embodiments, the FIASM is a compound that inhibits the enzyme aSMase, for example, a compound selected from the group consisting of amineptine, amitriptyline, amitripylinoxide, amoxapine, butriptyline, clomipramine, demexiptiline, desipramine, dibenzepin, dimetacrine, dosulepin, doxepine, imipramine, imipraminoxide, iprindole, lofepramine, melitracen, metapramine, nitroxazepine, nortriptyline, noxiptiline, pipofezine, propizenpine, protriptyline, quinupramine, tianeptine, trimipramine, and combinations thereof.

In some embodiments, the FIASM is selected from the group consisting of amitriptyline, clomipramine, desipramine, doxepine, imipramine, lofepramine, nortriptyline, protriptyline, trimipramine, and combinations thereof.

In some embodiments, the FIASM is selected from the group consisting of alimenazine, amlodipine, amitriptyline, astemizole, N-[(2-chlorophenyl)methyl]-1-[4-[[(2-chlorophenyl)methylamino]methyl]cyclohexyl]methanamine (AY 9944), benztropine, bepridil, camylofin, chloropromazine, chlorprothixene, clomipramine, clomiphene, cloperastine, cocaine, cyamemazine, cyclobenzaprine, cyproheptadine, desipramine, dibucaine, doxepin, drofenine, fendiline, fluoxetine, imipramine, maprotiline, mianserin, norfluoxetine, notriptyline, paroxetine, perhexiline, pimethixene, prochlorperazine, promazine, promethazine, propericiacine, protriptyline, quinacrine, sertraline, suloctidil, tamoxifen, terfenadine, thioproperazine, thioridazine, trifluoperazine, trifluopromazine, trihexylphenidyl, trimipramine, N-(6-Aminohexyl)-1-naphthalenesulfonamide (W-5), N-(6-aminohexyl)-5-chloro-1-naphthalenesulfonamide (W-7), and combinations thereof.

In some embodiments, the FIASM is selected from the group consisting of acetylsalicylic acid, aclacinomycin A, acrivastine, alaproclate, alimenazine, allylestrenol, alprenolol, alverine, amantadine, ambroxol, amiodarone, amitriptyline, amlodipine, amorolfine, antazoline, apomorphin, aprindine, astemizole, atovaquone, atropine, azacytidine, azaperone, azithromycin, barnidipine, benfluorex, benzbromarone, benztropine, bepridil, betaxolol, biperidene, bromhexine, bromocriptine, bromopride, bromperidol, brompheniramine, buclicine, bupivacain, bupropion, buspiron, butenafine, butorphanol, calcipotriol, camylofin, carbamazepine, carbenoxolone, carbetapentane, carvedilol, cepharanthine, chloropyramine, chloroquine, chlorotrianisene, chlorpheniramine, chlorpromazine, chlorprothixene, chlorquinaldol, cibenzoline, cilnidipine, cinnarizine, cisapride, citalopram, clarithromycin, clebopride, clemastine, clenbuterol, clofazimine, clomiphene, clomipramine, clonidine, cloperastine, cloricromen, clozapine, cocaine, colcemide, colchicine, conessine, cyamemazine, cyclazocine, cyclobenzaprine, cyclofenil, cycloheximide, cyclopentolate, cypermethrin, cyproheptadine, daunorubicin, desipramine, desloratadine, desogestrel, dexamethasone, dextromethorphan, diazepam, dibenzosuberane, dibucaine, dicyclomine, dienestrol, dilazep, diltiazem, dimebon, diosmin, diphenhydramine, diphenylpyralin, dirithromycin, disopyramide, D-mannitol, domperidone, donepezil, doxepin, doxorubicin, drofenine, droperidol, dutasteride, emetine, encainide, enoxolone, epinastine, erythromycine, etomidate, fantofarone, fendiline, fenfluramine, fenofibrate, fenspiride, fexofenadine, fipexide, flavoxate, flecainide, flufenamic acid, flunarizine, fluoxetine, flupenthixol, fluphenazine, flupirtine, fluspirilene, fluvoxamine, fosinopril, fulvestrant, fusidic acid, gabapentin, gentisic acid, glucose, haloperidol, harmine, hbuprofen, hdarubicin, hydrocortisone, hydroquinine, hydroxyurea, hydroxyzin, imidodibenzyle, imipramine, indomethacin, isoxsuprine, ketotifen, lamotrigine, lercanidipine, leukomethylene blue, lidocaine, L-leucine methyl ester, lofepramine, loperamide, loratadine, lynestrenol, maprotiline, mebeverine, mebhydrolin, mecamylamine, meclofenamic acid, mefenamic acid, memantine, mepacrine, mesoridazine, methapyrilene, metoclopramide, mianserin, mibefradil, mifepristone, mirtazapine, mitotane, moexipril, montelukast, moxisylyte, N-(6-Aminohexyl)-1-naphthalenesulfonamide (W-5), N-(6-aminohexyl)-5-chloro-1-naphthalenesulfonamide (W-7), N-[(2-chlorophenyl)methyl]-1-[4-[[(2-chlorophenyl)methylamino]methyl]cyclohexyl]methanamine (AY 9944), naphazoline, naproxen, nelfinavir, N-Methyl-5H-pyrido[3,4-b]indole-2-carboxamide (FG-7142), norfluoxetine, nortriptyline, noscapine, opipramol, orphenadrine, oxeladin, oxolamine, oxybutynine, oxymetazoline, oxyphencyclimine, papaverine, paraxanthine, paroxetine, penfluridol, pergolide, perhexiline, perphenazine, phenothrin, phenserine, phentolamine, phenylmethylsulfonyl fluoride, phenytoin, pimethixene, pimozide, pipamperone, pirarubicin, pirenperone, pranlukast, pridinol, prochlorperazine, procyclidine, profenamine, progesterone, promazine, promethazin, propafenone, proparacaine, propericiacine, propranolol, protriptyline, putrescine, pyrilamine, quetiapine, quinacrine, quinine, raloxifene, reboxetine, repaglinide, reserpine, retinol, rifabutin, rimantadine, ritanserin, rolipram, ropinirole, roxithromycin, salmeterol, selegeline, sertindole, sertraline, sibutramin, S-methylisothiourea, solasodine, sparteine, spiperone, spiramycin, stanozolol, sulindac, suloctidil, sulpiride, tacrine, tamoxifen, telmisartan, terfenadine, tetracaine, thiocarlide, thioproperazine, thioridazin, tiagabine, tibolone, tirofiban, tofisopam, tomatidine, tramadol, trifluoperazine, trifluperidol, triflupromazine, trihexyphenidyl, trimipramine, tripelennamine, triprolidine, tulobuterol, uridine, venlafaxine, verapamil, vinblastine, vincamine, vincristine, vinpocetine, warfarin, xylometazoline, yohimbin, zafirlukast, zolantidine, (S)-3-Methyl-2-phenyl-N-(1-phenylpropyl)-4-quinolinecarboxamide (SB-222200), 10-hydroxyimipramine, 2-hydroxyimipramine, 6-hydroxydopamine, and combinations thereof.

Studies characterizing the potency of FIASMs, for example, many TCAs demonstrate that there is variability in the residual aSMase activity after conventional treatment with a TCA compound. In such studies, the lower the residual activity the greater the potency of the compound. For example, following treatment with amitriptyline there is 11.7% residual activity, while imipramine has 30.6%, and haloperidol has 86.1% activity remaining. Hence, amitriptyline is more potent than imipramine which is far more potent than haloperidol. See Kornhuber J, Tripal P, Reichel M, Terfloth L, Bleich S et al. (2008) Identification of new functional inhibitors of acid sphingomyelinase using a structure-property-activity relation model. J Med Chem 51: 219-237.

Table 1, below, provides data reporting residual aSMase activity of a wide array of FIASMs, some of which are TCAs, as well as other agents, e.g., Carvedilol, which is an alpha/beta adrenergic antagonist, has a score of ˜22, better than imipramine (32.6). According to the instant disclosure, the definition of agents that are FIASMs are those with the ability to block residual aSMase activity to a level that is about or below 35%. Selecting from among various FIASMs it is possible to choose more or less potent drug options for use according to the skin protective composition s and methods hereof.

Taking into account drug potency, dosages of a FIASM can be in the range of 5 mg to 100 or more mg/dose (wherein dose is approximate and accomplished based on amount of the composition applied). In an aspect, the dosage of imipramine can be 5, 10, 15, 20, 25, 50, 75, 100, 200, or 300 mg or more total or any amount in between. In another aspect, dosage of amitriptyline which is approximately three times more potent than imipramine (see Table I) would be approximately 1 mg to 50 mg or more/dose.

In accordance with the various embodiments, the concentration of the FIASM in a skin protective composition can be in a range from about 0.1% by weight based on the weight of the composition up to and including about 25% by weight, alternatively from about 1% to about 10%, alternatively from about 0.1% to about 2%, alternatively from about 0.5% to about 2%, alternatively from about 0.1% to about 5%, alternatively from about 0.5% to about 5%, alternatively from about 2% to about 5%, by weight, or any suitable combination, sub-combination, range, or sub-range thereof by weight, based on the total weight of the composition. One of ordinary skill in the art, however, will appreciate that other ranges are within the scope of the invention.

In some particular embodiments, the skin protective composition includes an FIASM, for example, imipramine, present in a range from about 2% to about 5% by weight based on the weight of the composition. In some particular embodiments, the skin protective composition also includes propylene glycol present in a range from about 1% to about 25% by weight based on the weight of the skin care composition. Similarly, it would be expected that amitriptyline, for example, would be employed at lower concentrations.

Accordingly, selection of dosage and concentration for each of the various FIASMs may vary according to their demonstrated activity in at least an assay representative of the data shown in table 1, among other factors. In all embodiments, it is the inclusion of the FIASM in a suitable dermatological carrier for topical application to address photosensitivity that is the key unexpected use of FIASM compounds, irrespective of concentration, amount, application frequency, product form, or inclusion of carrier ingredients.

In various embodiments, an effective dose of a FIASM for reducing or preventing photosensitivity demonstrates inhibition of acid sphingomyelinase, reduces or eliminates inflammation, reduces or eliminates microvesicle particle release, reduces or eliminates erythema, reduces or eliminates an immunosuppressive effect from topical photodynamic therapy, or a combination thereof.

TABLE 1

| | | | | | Incu- | Resid- |
|---|---|---|---|---|---|---|
| Com- | | | | | bation | ual |
| pound | | | | Acid/ | time | ASM |
| No. | Name | Set | CID | Base | (h) | Activity |
| 2008-Paper | | | | | | |
| 6 | Amitriptyline | 1 | 2160 | 1 | 0.5 | 11.7 |
| 12 | Clomipramine | 1 | 2801 | 1 | 0.5 | 21.8 |
| 25 | Perhexiline | 1 | 4746 | 1 | 0.5 | 8.5 |
| 32 | Thioridazin | 2 | 5452 | 1 | 6 | 10.4 |
| 35 | Trimipramine | 1 | 5584 | 1 | 0.5 | 13.8 |
| 40 | Alverine | 2 | 3678 | 1 | 24 | 21.7 |
| 41 | Amlodipine | 1 | 2162 | 1 | 0.5 | 12.0 |
| 43 | Astemizole | 2 | 2247 | 3 | 6 | 14.3 |
| 45 | Benztropine | 1 | 6832 | 1 | 0.5 | 12.7 |
| 46 | Bepridil | 2 | 2351 | 3 | 24 | 27.1 |
| 47 | Bromhexine | 1 | 2442 | 1 | 48 | 102.8 |
| 50 | Camylofin | 2 | 5902 | 3 | 24 | 21.7 |
| 55 | Clomiphene | 1 | 1548955 | 1 | 0.5 | 13.0 |
| 56 | Cloperastine | 2 | 2805 | 1 | 24 | 26.7 |
| 59 | Cyclobenzaprine | 1 | 2895 | 1 | 0.5 | 26.2 |
| 60 | Cyproheptadine | 1 | 2913 | 1 | 0.5 | 22.2 |
| 64 | Drofenine | 2 | 3166 | 1 | 24 | 20.8 |
| 67 | Flunarizine | 1 | 941361 | 1 | 48 | 32.7 |
| 71 | Lofepramine | 1 | 3947 | 1 | 24 | 19.2 |
| 74 | Mibefradil | 2 | 60663 | 3 | 6 | 20.8 |
| 79 | Penfluridol | 1 | 33630 | 1 | 6 | 22.0 |
| 80 | Pimethixene | 1 | 4822 | 1 | 24 | 16.5 |
| 81 | Pimozide | 2 | 16362 | 1 | 24 | 30.6 |
| 84 | Promazine | 1 | 4926 | 1 | 0.5 | 33.6 |
| 85 | Protriptyline | 1 | 4976 | 1 | 0.5 | 12.7 |
| 98 | Dicyclomine | 2 | 3042 | 1 | 24 | 18.6 |
| 102 | Fendiline | 1 | 3336 | 1 | 0.5 | 25.2 |
| 104 | Fluoxetine | 1 | 3386 | 1 | 0.5 | 13.0 |
| 109 | Maprotiline | 1 | 4011 | 1 | 0.5 | 13.5 |
| 110 | Mebeverine | 2 | 4031 | 1 | 24 | 31.8 |
| 113 | Norfluoxetine | 1 | 4541 | 1 | 0.5 | 22.5 |
| 114 | Nortriptyline | 1 | 4543 | 1 | 0.5 | 13.3 |
| 118 | Paroxetine | 1 | 43815 | 1 | 0.5 | 31.7 |
| 120 | Promethazin | 1 | 4927 | 1 | 0.5 | 32.2 |
| 124 | Sertraline | 1 | 68617 | 1 | 0.5 | 12.3 |
| 127 | Terfenadine | 1 | 5405 | 1 | 0.5 | 21.8 |
| 129 | Triflupromazine | 1 | 5568 | 1 | 0.5 | 29.5 |
| New set | | | | | | |
| 138 | Amiodarone | 3 | 2157 | 1 | 24 | 14.5 |
| 141 | Aprindine | 3 | 2218 | 3 | 6 | 27.5 |
| 143 | AY9944 | 3 | 9705 | 3 | 0.5 | 22.1 |
| 149 | Biperidene | 3 | 2381 | 1 | 24 | 26.2 |
| 160 | Carvedilol | 3 | 2585 | 1 | 24 | 22.4 |
| 161 | Cepharanthine | 3 | 360849 | 3 | 6 | 9.2 |
| 170 | Clemastine | 3 | 26987 | 1 | 24 | 12.6 |
| 172 | Clofazimine | 3 | 2794 | 1 | 48 | 23.7 |
| 175 | Conessine | 3 | 441082 | 3 | 24 | 20.8 |
| 181 | Desipramine | 3 | 2995 | 1 | 0.5 | 15.6 |
| 182 | Desloratadine | 3 | 124087 | 3 | 0.5 | 21.9 |
| 196 | Emetine | 3 | 10219 | 3 | 24 | 0.4 |
| 208 | Flupenthixol | 3 | 5281881 | 3 | 0.5 | 18.2 |
| 222 | Imipramine | 3 | 3696 | 1 | 0.5 | 32.6 |
| 227 | Loperamide | 3 | 3955 | 1 | 24 | 24.4 |
| 249 | Perphenazine | 3 | 4748 | 3 | 0.5 | 20.1 |
| 258 | Profenamine | 3 | 3290 | 1 | 0.5 | 29.7 |
| 285 | Tamoxifen | 3 | 2733526 | 1 | 6 | 4.1 |
| 291 | Tomatidine | 3 | 65576 | 1 | 24 | 15.8 |
| 292 | Trifluoperazine | 3 | 5566 | 3 | 6 | 8.3 |
| 306 | Zolantidine_ | 3 | 91769 | 3 | 6 | 21.6 |
| | (SKB41) | | | | | |

The data were obtained in a cell-based study by adding 10 micromolar aliquots of the above agents to PC12 cells for the indicated amounts of time (usually 0.5, 6, or 24 hours). Cells were lysed and aSMase activity measured using [choline methyl-14C] sphingomyeline. The amount of aSMase activity following the treatment of the agent was compared to vehicle without any FIASM. Agents that inhibit aSMase activity to 35% or lower residual level of aSMase activity are considered exhibiting FIASM activity. The data for the listed compounds (n=276) is based on the publication by Kornhuber et al. 2008, including compounds number n=101 combined with an additional set of compounds (n=175) Kornhuber J, Muehlbacher M, Trapp S, Pechmann S, Friedl A, Reichel M, Mühle C, Terfloth L, Groemer T W, Spitzer G M, Liedl K R, Gulbins E, Tripal P. Identification of novel functional inhibitors of acid sphingomyelinase. PLOS One. 2011; 6 (8): e23852. doi: 10.1371/journal.pone.0023852. Epub 2011 Aug. 31. PMID: 21909365; PMCID: PMC3166082. The numbering of compounds is identical to the previous publication for the first 101 compounds. CID=PubChem Compound ID. Set: 1=experimental values from Kornhuber et al. 2008, 2=corrected experimental values of the previously published compounds; 3=newly investigated compounds, Acid/Base: 1=monobase, 2=monoacid, 3=bibase, 4=zwitter. Mean residual activity of ASM is given as % of the corresponding control values.

In some embodiments the FIASM is a compound selected the group consisting of Amitriptyline, Clomipramine, Perhexiline, Thioridazin, Trimipramine, Alverine, Amlodipine, Astemizole, Benztropine, Bepridil, Bromhexine, Camylofin, Clomiphene, Cloperastine, Cyclobenzaprine, Cyproheptadine, Drofenine, Flunarizine, Lofepramine, Mibefradil, Penfluridol, Pimethixene, Pimozide, Promazine, Protriptyline, Dicyclomine, Fendiline, Fluoxetine, Maprotiline, Mebeverine, Norfluoxetine, Nortriptyline, Paroxetine, Promethazin, Sertraline, Terfenadine, Triflupromazine, Amiodarone, Aprindine, AY9944, Biperidene, Carvedilol, Cepharanthine, Clemastine, Clofazimine, Conessine, Desipramine, Desloratadine, Emetine, Flupenthixol, Imipramine, Loperamide, Perphenazine, Profenamine, Tamoxifen, Tomatidine, Trifluoperazine, Zolantidine (SKB41), and combinations thereof, wherein the FIASM demonstrates inhibition of aSMase activity to 35% or lower residual activity.

### UV Filters

In accordance with the various embodiments related to photosensitivity, the methods and compositions according to the disclosure may include use of inorganic/mineral based and/or organic UV filters. The organic UV filters, when present, are typically provided in an oil phase of an emulsion form of skin protective composition.

In some embodiments, the one or more mineral UV filters are selected from the group consisting of titanium dioxide, zinc oxide, iron oxides, cerium oxides, zirconium oxides, and combinations thereof. In some embodiments, the at least one mineral UV filter comprises zinc oxide, a zinc oxide derivative, titanium dioxide, a titanium dioxide derivative, or combinations thereof. In some embodiments, the one or more organic UV filters may be hydrophilic and/or lipophilic. The organic UV filter may be solid or liquid. The terms “solid” and “liquid” mean solid and liquid, respectively, at 25° C. under 1 atm.

The organic UV filter can be selected from the group consisting of anthranilic compounds; dibenzoylmethane compounds; cinnamic compounds; salicylic compounds; camphor compounds; benzophenone compounds; diphenylacrylate compounds; triazine compounds; benzotriazole compounds; benzalmalonate compounds; benzimidazole compounds; imidazoline compounds; bis-benzoazolyl compounds; p-aminobenzoic acid (PABA) compounds; methylenebis(hydroxyphenylbenzotriazole) compounds; benzoxazole compounds; dimers derived from α-alkylstyrene; 4,4-diarylbutadienes compounds; guaiazulene and derivatives thereof; rutin and derivatives thereof; flavonoids; bioflavonoids; oryzanol and derivatives thereof; quinic acid and derivatives thereof; and combinations thereof.

### Photosensitizing Agents

In accordance with the various embodiments related to topical photodynamic therapy, the methods and compositions according to the disclosure may include use of photosensitizing agents. In some such embodiments, the photosensitizing agents may be included with the FIASM in the skin protective composition.

Appropriate photosensitizing agents for PDT may include, but are not limited to, porphyrins, chlorins, and dyes. Preferred photosensitizing agents have changed over the years and are often classified as first-generation, second-generation, or third-generation. The most common first-generation photosensitizing agent is photofrin, a mixture of porphyrin dimers and oligomers. First-generation photosensitizing agents are currently used less frequently due to side effects that include skin sensitivity and their weak absorption at 630 nm.

Two common second-generation photosensitizing agents are aminolevulinic acid (ALA) and its methyl ester form, methyl aminolevulinate (MAL). Both can be topically applied and have some selectivity for precancerous and cancerous cells, although MAL is more lipophilic and absorbed by deeper skin tissues than ALA. These agents are converted into the highly reactive protoporphyrin IX (PpIX). Notably, rapidly proliferating cells, such as cancer cells, convert more ALA to PpIX in their mitochondria than their non-transformed counterparts in the epidermis. When neoplastic cells are exposed to light at various wavelengths (classically between 570 and 670 nm), ROS are created which then damage and destroy target cells. In clinical practice, both red (most commonly 630 nm) and blue (410-420 nm) wavelength light sources are utilized. Moreover, “daylight PDT” is also employed, using natural sunlight to activate these photosensitizing agents. Finally, third-generation photosensitizing agents are antibody-directed and were developed to have a strong affinity for tumor cells, causing less damage to the surrounding tissues.

In some embodiments, photosensitizing agents may comprise one or more of aminolevulinic acid, methyl aminolevulinate, 5-aminileuvulinic acid, porphyrin, protoporfin IX, purlytin, verteporfin, HPPH, temoporfin, methylene blue, photofrin, hematoporphyrin, Talaporfin, benzopophyrin derivative monoacid, Lutetium texaphyrin, metallophthalocyanine, metallo-naphthocyaninesulfobenzo-porphyrazine, metallo-naphthalocyanines, zinc tetrasulfophthalocyanine, bacteriochlorins, metallochlorins, chlorine derivative, Tetra(m-hydroxyphenyl)chlorin (mTHPC), pheophorbide, dibromofluorescein (DBF), IR700DX, naphthalocyanine, porphyrin derivative, or combinations thereof. A listing of photosensitive chemicals may be found in Kreimer-Bimbaum, Sem. Hematol. 26:157-73, 1989 (incorporated herein by reference) and in Redmond and Gamlin, Photochem. Photbiol. 70 (4): 391-475 (1999).

In accordance with some embodiments according to the disclosure, the amount of photosensitizer that may be employed in a skin care composition is dependent on factors such as the specific type of photosensitizer used and the type of activation energy source according to what is known in the art. Generally, the photosensitizer component of the skin protective composition s herein may comprise from about 0.0001% to about 100%, or from about 0.001% to about 50%, or from about 0.01% to about 25%, or from about 0.1% to about 10%, by weight, of photosensitizer, or any suitable combination, sub-combination, range, or sub-range thereof by weight, based on the total weight of the composition. One of ordinary skill in the art, however, will appreciate that other ranges are within the scope of the invention.

In some embodiments, the photosensitizer component may comprise up to about 100% of the photosensitizing agents comprising 5-aminileuvolinic acid.

### Dermatologically Acceptable Carriers and Actives

In accordance with the various embodiments according to the disclosure, the methods and compositions may include use of dermatologically acceptable carriers that may include water and one or more ingredients that may include one or more solvents. In some embodiments, the carrier is a dermatologically acceptable carrier. In some embodiments, the carrier is a dermatologically acceptable carrier.

Generally, in accordance with the various embodiments, compositions according to the disclosure may include a dermatologically acceptable carrier. The total amount of the dermatologically acceptable carrier in compositions may be from about 1% to about 99%, based on the total weight of the composition. Thus, the dermatologically acceptable carrier may be present, by weight, based on the total weight of the composition, from about 1, 2, 3, 4, 5, 6, 7, 8, 9, 10, 15, 20, 25, 30, 35, 40, 45, 50, 55, 60, 65, 70, 75, 80, 85, 90, 95 to about 99 percent, by weight, including increments and ranges therein and there between. One of ordinary skill in the art, however, will appreciate that other ranges are within the scope of the invention.

In some embodiments, the dermatologically acceptable carrier includes water present in a range from about 1% to about 99%, based on the total weight of the composition. Thus, water may be present, by weight, based on the total weight of the composition, from about 1, 2, 3, 4, 5, 6, 7, 8, 9, 10, 15, 20, 25, 30, 35, 40, 45, 50, 55, 60, 65, 70, 75, 80, 85, 90, 95 to about 99 percent, by weight, including increments and ranges therein and there between. One of ordinary skill in the art, however, will appreciate that other ranges are within the scope of the invention.

The water used may be sterile demineralized water and/or a floral water such as rose water, cornflower water, chamomile water or lime water, and/or a natural thermal or mineral water.

The pH of the composition may be adjusted with pH adjusters to a pH in a range from about 3 to about 9, or from about 5 to about 8, or about 6.5 to about 7.5 by addition of a base (organic or inorganic) to the composition, for example sodium hydroxide, ammonia or a primary, secondary or tertiary (poly)amine, such as monoethanolamine, diethanolamine, triethanolamine, isopropanolamine or 1,3-propanediamine, or alternatively by addition of an inorganic or organic acid, advantageously a carboxylic acid, such as, for example, citric acid.

The dermatologically acceptable carrier can include in addition to water, water-soluble solvents. In some embodiments that may include water-soluble solvents, the composition is nevertheless free of and does not include or is free of any one or more of ethoxydiglycol, dimethyl isosorbide, triethyl citrate, isopropyl lauryl sarcosinate, propylene glycol, propanediol, or combinations thereof. In some particular embodiments, the skin protective composition does not include or is free of at least one or more of water-soluble solvents selected from the group consisting of monoalcohols and polyols, diols, glycols, glycol ethers and combinations thereof.

The term “water-soluble solvent” is interchangeable with the term “water-miscible solvent” and means a compound that is liquid at 25° C. and at atmospheric pressure (760 mmHg), and it has a solubility of at least 50% in water under these conditions. In some cases, the water-soluble solvent has a solubility of at least 60%, 70%, 80%, or 90%. Non-limiting examples of water-soluble solvents include, for example, glycerin, alcohols (for example, C1-30, C1-15, C1-10, or C1-4 alcohols), organic solvents, polyols (polyhydric alcohols), glycols (e.g., butylene glycol, caprylyl glycol, etc.), and a mixture thereof.

As examples of organic solvents, non-limiting mentions can be made of monoalcohols and polyols such as ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, propane diol, and glycerin. The organic solvents can be volatile or non-volatile compounds.

Further non-limiting examples of water-soluble solvents include alkanediols such as glycerin, 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

Polyhydric alcohols are useful. Examples of polyhydric alcohols include glycerin, ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof.

In some instances, the skin protective composition s of the disclosure include one or more glycols and/or one or more alcohols, for example, one or more water-soluble solvents selected from the group consisting of butylene glycol, caprylyl glycol, propanediol, glycerin, and a mixture thereof.

The total amount of the one or more water-soluble solvents can vary but is typically about 1 to about 40% by weight, based on the total weight of the skin protective composition. In some cases, the total amount of the one or more water-soluble solvents is about 1 to about 35% by weight, about 1 to about 30% by weight, about 1 to about 25% by weight, about 2 to about 40% by weight, about 2 to about 35% by weight, about 2 to about 30% by weight, about 2 to about 25% by weight, about 5 to about 40% by weight, about 5 to about 35% by weight, about 5 to about 30% by weight, about 5 to about 25% by weight, about 10 to about 40% by weight, about 10 to about 35% by weight, about 10 to about 30% by weight, about 10 to about 25% by weight, about 15 to about 40% by weight, about 15 to about 35% by weight, about 15 to about 30% by weight, or about 15 to about 25% by weight, based on the total weight of the skin protective composition.

In the case of emulsion-based treatments for photosensitivity that include oil soluble organic UV agents, the solvents may be present in one or both of an aqueous phase and/or an oil phase of the emulsion. Thus, in some embodiments, the methods and compositions may include use of ingredients selected from the group consisting of water based solvents such as humectants including glycols, UV active boosters and stabilizers, oils and oil based solvents, and combinations thereof. The methods and compositions may include use of one or a combination of actives or optional additives used in the dermatologic agents field, which does not affect the properties of the treatment methods or compositions according to the invention, such as vitamins, other skin care actives, fragrances, dyes, preservatives, vitamins, oils, essential oils, salts, neutralizing or pH-adjusting agents, and combinations thereof. In some embodiments, additional actives may include water, water-based solvents, oils, oil-based solvents, antimicrobials, antibiotics, redness reducers, antioxidants, emollients, and combinations thereof. In some specific examples, additional skin active include metronidazole, clindamycin, and combinations thereof.

In some particular photosensitivity embodiments, the methods and compositions may include use of dermatologically acceptable carriers that may include polyols, including, for example, glycerin, glycerol, glycols, such as caprylyl glycol, butylene glycol, propanediol, propylene glycol, isoprene glycol, dipropylene glycol, hexylene glycol and polyethylene glycols, monoethylene glycol, diethylene glycol, triethylene glycol, diethylene glycol, hexylene glycol, glycol ethers such as monopropylene, dipropylene, and tripropylene glycol (C₁-C₄) alkyl ethers, squalane, triacetin, sugars, such as glucose, xylitol, maltitol, sorbitol, sucrose pentaerythritol, inositol, pyrrolidone carboxylic acid, lactic acid, lithium chloride, acetamide monoethanolamine (MEA), sodium lactate, urea, dicyanamide, hyaluronic acid, aloe vera, seaweed extract, and combinations thereof present from about 1% to about 25%, or from about 2% to about 20%, or from about 3% to about 5% or any suitable combination, sub-combination, range, or sub-range thereof by weight, based on the total weight of the composition. One of ordinary skill in the art, however, will appreciate that other ranges are within the scope of the invention.

In some particular embodiments, the methods and compositions may include use of dermatologically acceptable carriers that include propylene glycol.

### Other Additives

In accordance with some embodiments, other additives useful for compositions and methods hereof include but are not limited to surfactants, stabilizing agents, dispersants, wetting agents, emulsifying agents, thickeners and rheology modifiers, film formers, waxes, moisturizers, emollients, vitamins, antioxidants, sunscreen/UV protection, analgesics, anti-inflammatory, hemostatics, perfumes, hydrophobic barrier constituents, anti-microbial, biologic trophic agents, enzymes, fiber and combinations thereof, all by way of example.

In accordance with some embodiments, the skin protective composition s and methods may include one or more dermal penetrating enhancers, including microemulsions, alcohols and polyols (e.g., ethanol, glycerol, propylene glycol), surfactants, fatty acids (e.g., Oleic acid), amines and amides (e.g., Azone, N-methylpyrrolidone), terpenes (e.g., limonene) sulfoxides (e.g., dimethylsulfoxide-DMSO), esters (e.g., isopropyl myristate), and combinations of these, to promote the penetration of the FIASM through the skin. In some embodiments, the compositions and methods include use of propylene glycol.

In accordance with the various embodiments wherein the composition comprises one or more additives, each such additive may be present from about 0.0001% to about 99%, or from about 0.01% to about 50%, or from about 0.5% to about 10% by weight, based on the total weight of the composition.

More generally, the skin protective composition s according to the disclosure include ingredients that are present in amounts by weight, typically based on the weight of the composition, wherein the ingredients may be present at a concentration, by weight, of from about 0.001% to about 100%. Thus, in various embodiments, an ingredient may be present in a composition in a weight percent amount from about 0.0001, to about 0.001, to about 0.01, to about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98 to about 99 percent by weight, including increments and ranges thereof and there between.

In accordance with the various photosensitivity embodiments according to the disclosure, the methods and compositions may include formulation of a therapeutically effective amount of a FIASM, alone or in combination with one or more of acceptable carriers, UV actives/sunscreen actives, solvents and other suitable ingredient, for treatment and/or prophylaxis. According to the disclosure, imipramine is employed in the skin protective composition. In some embodiments, alternative treatments and compositions may include a FIASM, for example, a FIASM selected from the group consisting of amineptine, amitriptyline, amitripylinoxide, amoxapine, butriptyline, clomipramine, demexiptiline, desipramine, dibenzepin, dimetacrine, dosulepin, doxepine, imipramine, imipraminoxide, iprindole, lofepramine, melitracen, metapramine, nitroxazepine, nortriptyline, noxiptiline, pipofezine, propizenpine, protriptyline, quinupramine, tianeptine, trimipramine, and combinations thereof.

The therapeutically effective amount or dosage of the FIASM used in the methods as disclosed herein applied to mammals (e.g., humans) can be determined with consideration of individual differences in age, weight, sex, other drugs administered, and the judgment of the treating clinician. Variations in dosage levels can be adjusted using standard empirical routes for optimization. The particular dosage of a skin protective composition to be administered to the patient may depend on a variety of considerations (e.g., the severity of the skin condition symptoms), the age and physical characteristics of the subject and other considerations known to those of ordinary skill in the art. Dosages can be established using clinical approaches known to one of ordinary skill in the art.

In some embodiments, the amount or concentration of the FIASM is demonstrated to inhibit aSMase.

The skin protective composition s as disclosed herein are prepared for transdermal (e.g., topical) administration. The delivery form of the compositions including a FIASM may be a cream, a gel, an ointment, or a spray or other non-limiting form as mentioned herein, and may be anhydrous, a water-in-oil emulsion, or an oil-in-water emulsion. In some embodiments, the skin protective composition is a skin protective composition.

The duration of treatment with any composition provided herein including a FIASM can be any length of time from as short as one treatment for topical photodynamic therapy, or one day to as long as the life span of the subject (e.g., from days to weeks to months to years) for photosensitivity. For example, the skin protective composition may be applied on a daily basis, and in particular prior to sun exposure for photosensitivity. Alternatively for photosensitivity, the compositions can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

Regarding its use for enhancing the efficacy of PDT by inhibiting the unwanted immunosuppressive effects and acting as a nociceptive, the therapeutic treatment could be used at the time of application of the photosensitizer, or afterwards, to include application after the light treatment.

The skin protective composition s described herein comprising a FIASM can be packaged in a suitable container labeled, for example, for use as a therapy to treat cancer or any of the methods disclosed herein. Accordingly, packaged products (e.g., sterile containers containing the composition described herein and packaged for storage, shipment, or sale at concentrated or ready-to-use concentrations) and kits, including at least one FIASM as described herein and instructions for use, are also within the scope of the disclosure. A product can include a container (e.g., a vial, jar, bottle, bag, or the like) containing the composition described herein. In addition, an article of manufacture further may include, for example, packaging materials, instructions for use, applicators, diluents or other control reagents for treating or monitoring the condition for which prophylaxis or treatment is required.

### EXAMPLES

The following Examples are provided for illustrative purposes only and are not intended to be limiting.

### Example 1: Xeroderma Pigmentosum a Deficiency Results in Increased Generation of MVPs in Response to Ultraviolet B Radiation In Vitro

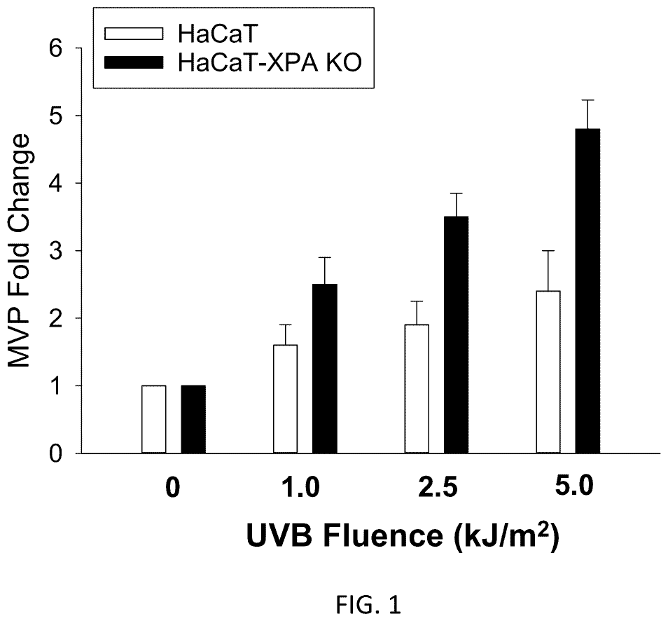

HaCaT cells and HaCaT XPA deficient cells received no treatment (NT) or 1 KJ/m², 2.5 KJ/m², or 5 KJ/m² UVB radiation. After 4 hours, the supernatant was removed and the MVP concentration was measured. The numbers of MVP shown in FIG. 1 were normalized to cell count and NT. Data in FIG. 1 show the mean+S.E. fold change of at least three experiments. Groups were compared using One-Way ANOVA. (P<0.01) was considered statistically significant. * denotes changes between NT and UVB. #denotes changes between HaCaT and HaCaT XPA deficient cells.

FIG. 1 shows that UVB generates increased MVP release in vitro in HaCaT XPA deficient cells compared to HaCaT cells lacking XPA deficiency.

### Example 2: Xeroderma Pigmentosum a Deficiency Results in Increased Generation of MVPs in Response to Ultraviolet B Radiation In Vivo

Groups of 8-13 wild-type (C57BL/6) and XPA KO mice received no treatment (NT) or 1 KJ/m², 2.5 kJ/m², or 5 KJ/m² of UVB to their dorsal back skin. In some experiments, mice were treated with topical application of the PAFR agonist carbamoyl-PAF (CPAF; 50 μL of 100 μM) or the phorbol ester 12-O-tetradecanoylphorbol-13-acetate (TPA; 50 μL of 100 μM) in 90% ethanol: 10% DMSO vehicle. For the data in FIG. 2, triplicate skin biopsies were obtained and weighed four hours post-treatment, and MVP was measured. FIG. 2 shows the resulting normalized MVP concentrations in the skin tissue. For the data in FIG. 3, plasma was obtained and MVP was measured four hours post-treatment. FIG. 3 shows the resulting normalized MVP concentrations in the plasma. Data are the mean±S.E. fold change of MVP count. Groups were compared using One-Way ANOVA and Tukey's post-hoc test. Differences in samples were considered significant if the P value was less than 0.05. P<0.05 (#) changes between WT and XPA KO mice; P<0.05 (*) changes between NT and UVB treatment.

FIG. 2 and FIG. 3 show that exposure to UVB irradiation generates increased MVP release in vivo in XPA KO mice. Moreover, TPA or CPAF treatment resulted in similar levels of MVP generation in WT compared to XPA KO mice.

### Example 3: Imipramine Reduces UVB-Mediated MVP Formation

Groups of 6-8 wild-type and XPA KO mice were either received 50 μl of 90% ethanol:10% DMSO, vehicle (VEH), imipramine 500 μM only, 5 KJ/m² UVB followed by vehicle, or UVB+imipramine 500 μM and were analyzed after 4 hours of post-treatment. For the data shown in FIG. 4, triplicate skin biopsies were obtained and MVP was measured. For the data shown in FIG. 5, plasma was obtained and MVP was measured. Data is shown as mean±S.E. of fold change in MVP count. P<0.05 (*) between VEH and UVB. Statistically significant P<0.01 ($) changes between UVB only and UVB+imipramine in both WT and XPA KO mice and P<0.05 between UVB group in WT and XPA KO mice.

FIG. 4 and FIG. 5 show that the FIASM imipramine inhibits UVB-induced MVP release in XPA KO mice.

### Example 4: Effect of Topical Imipramine on UVB Irradiation-Induced Erythema and Cytokine Responses of XPA KO Mice in Comparison to Wild-Type Mice

Groups of 6-8 mice underwent erythema measurements with a Mexameter® device before irradiation with various fluences (1.0, 2.5, and 5.0 kJ/m²) of UVB or no UVB (sham), and four hours later erythema was measured to determine the cutaneous erythema and cytokine responses of UVB irradiation on wild-type vs XPA KO mice. Similarly, triplicate skin biopsies were obtained and mRNA isolated, and quantitative reverse transcription polymerase chain reaction (qRT-PCR) used to quantify mRNA levels of cytokines TNF [symbol] and IL-6. The housekeeping gene GAPDH was used as control. As shown in FIG. 6, UVB irradiation resulted in enhanced erythema responses in XPA KO mice over wild-type mice. The data is shown as the mean±S.E. of fold change in baseline erythema levels. * Denotes statistically significant (P<0.05) differences between UVB- and sham-treated mice. #Denotes statistically significant (P<0.05) differences between XPA KO and wild-type erythema responses.

Mice were treated similarly as in FIG. 6, except that 100 μL of either 90% ethanol:10% DMSO vehicle (VEH) or 500 μM imipramine was applied immediately following irradiation with 5 kJ/m² UVB to determine the effects of topical imipramine on UVB-induced erythema responses. FIG. 7 shows the results, indicating that the augmented UVB-induced erythema responses characteristic of photosensitive XPA KO mice were blocked by imipramine. The data is shown as the mean±S.E. of fold change in baseline erythema levels. * Denotes statistically significant (P<0.05) differences between UVB- and sham-treated mice

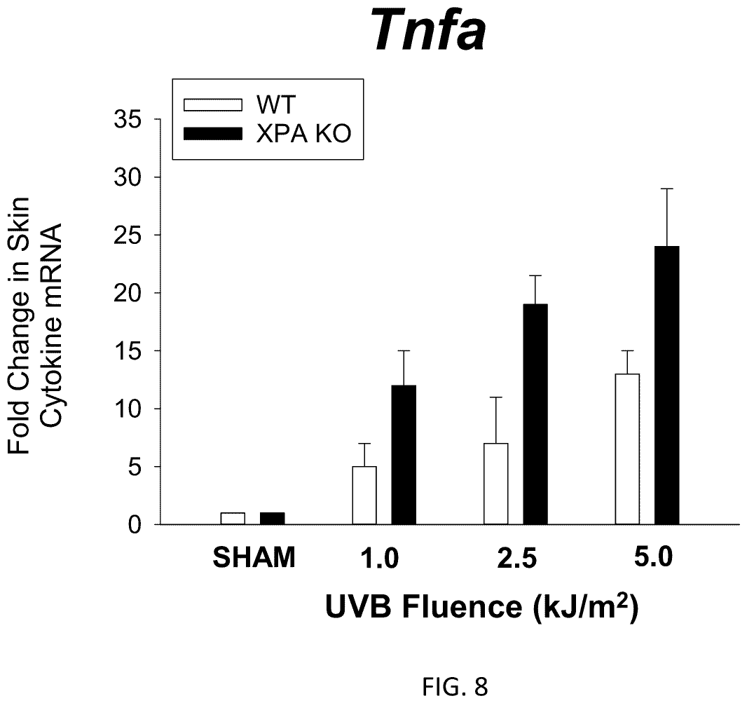

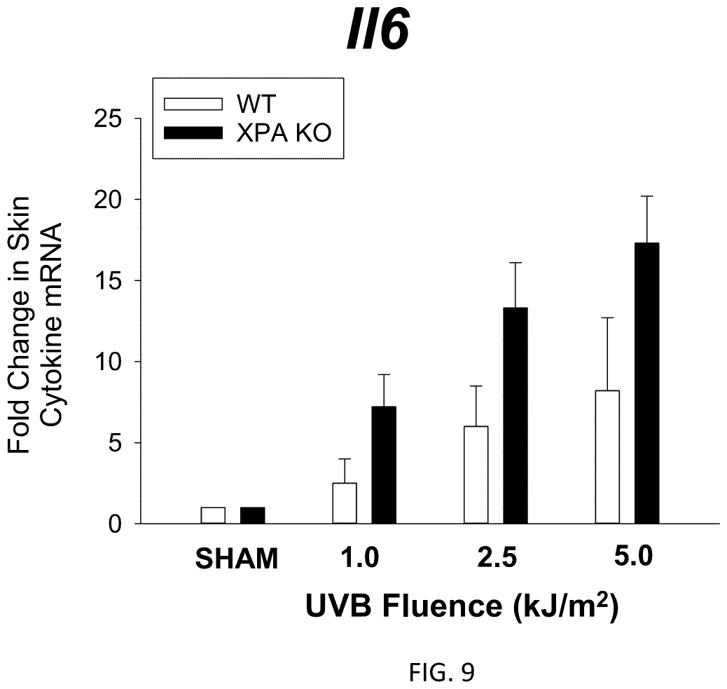

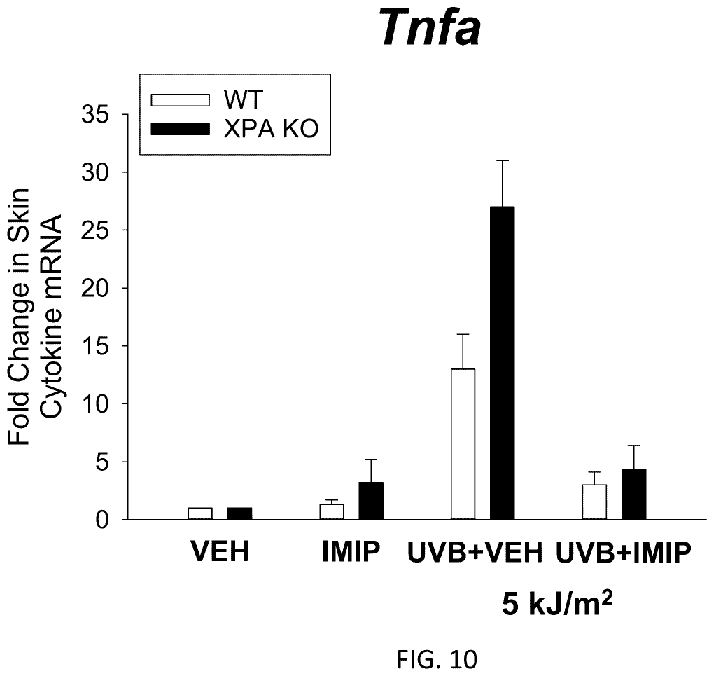

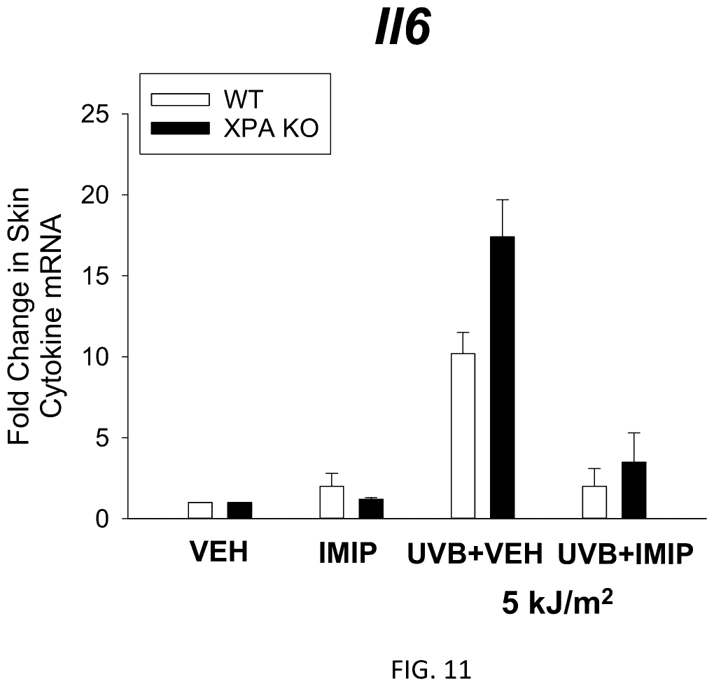

Regarding UVB-induced cytokine release, FIG. 8 and FIG. 9 show the results of qRT-PCR, demonstrating that UVB irradiation results in increased TNFα and IL-6 gene expression levels in XPA KO vs wild-type mice. Similar to the erythema levels, topical imipramine applied post-UVB blocks the induction of these pro-inflammatory cytokines. The data is shown as the mean±S.E. of fold change in baseline erythema levels. * Denotes statistically significant (P<0.05) differences between UVB- and sham-treated mice. FIG. 12 depicts mean±S.E. of fold change of in levels of MVP, erythema and cytokine levels in groups of at least six wild-type, aSMase KO and XPA KO mice.

Finally, FIG. 13 shows that both pre-treatment and post-treatment with imipramine blocks UVB-induced MVP formation in human skin explants. Skin explants were treated with 100 μL of 90% ethanol:10% DMSO vehicle (VEH) or 500 μM imipramine, or 2,500 J/m² UVB. In some experiments the vehicle or imipramine was applied 30 minutes before the UVB treatment, and others was immediately after the UVB irradiation. Four hours post-UVB triplicate skin biopsies were obtained and MVP quantified. These data are the mean±S.E. of fold change in MVP levels from untreated (sham) values from four separate experiments. * Denotes statistically significant (P<0.05) differences between UVB- and sham-treated skin. #Denotes statistically significant (P <0.05) differences between vehicle- and imipramine-treated MVP levels.

Together, these results indicate that the enzyme aSMase is essential for MVP release and that targeting this enzyme blocks the exaggerated UVB-mediated responses associated with photosensitivity. Moreover, these results confirm that topical imipramine can be used on human skin to either prevent, or treat UVB-generated MVP release.

### Example 5: PDT Increases MVP Release

PDT-generated MVP release in HaCaT keratinocytes and human skin explants was evaluated. For the data shown in FIG. 14, HaCaT keratinocytes were treated with sham (no treatment), 0.1% ethanol vehicle control (VEH), 100 nM TPA, 100 nM CPAF, 1 mM 5-ALA for 4 hours, or 5-ALA followed by various fluences (2.5-20 J/cm²) of blue light or blue light alone. Levels of MVPs released into supernatants were measured at 4 hours. Similar tests on human fibroblasts which lack PAFRs (data not shown) resulted in robust increases in MVP levels in response to PDT but not the PAFR agonist CPAF, which provided further evidence that the PAFR is not needed to generate MVP in response to PDT stimulus. For the data shown in FIG. 15, human skin explants were treated with sham, 100 μL vehicle (90% ethanol+10% DMSO) (VEH), 1 μM TPA, 1 UM CPAF, 1 mM 5-ALA for 4 hours, blue light or 5-ALA+blue light at 20 J/cm². After four hours, duplicate 6-mm punch biopsies were obtained, weighed, and MVPs were measured. The data are the mean±SE from 3-4 separate experiments conducted in duplicate. * Denotes statistically significant (P <0.05) changes from sham values. Increasing levels of blue light radiation+5-ALA produced increasing MVP generation.

FIG. 14 shows that PDT resulted in the release of MVP in a dose- and time-dependent manner in the HaCaT keratinocyte cell line, and FIG. 15 shows that treatment of human skin explants by PDT also triggers MVP release.

### Example 6: Imipramine Reduces PDT Generation of MVP

The ability of imipramine to control PDT-mediated MVP release was evaluated. For the data shown in FIG. 16, HaCaT keratinocytes were treated as in FIG. 14, with the exception that in some experiments 50 M of the FIASM imipramine (IMIP) was added immediately after the PDT. Supernatants were collected at 4 hours and MVP measured. The data are the mean±SE MVP fold change from sham-treated values. For the data shown in FIG. 17, human skin explants were treated as in FIG. 15, with the exception that in some experiments 100 μL of 5 mM IMIP was added post-PDT. MVP were measured in duplicate 6-mm punch biopsies obtained 4 hours post-treatment. The data are the mean±SE MVP fold change from sham-treated values from four separate experiments. * Denotes statistically significant (P<0.05) differences between sham- or vehicle-treated skin. #Denotes statistically significant (P<0.05) differences between vehicle- and imipramine-treated MVP levels.

The role of aSMase in PDT-mediated MVP release was evaluated in a murine system. For the data shown in FIG. 18, wild-type mice were treated with either sham, 100 μl of 5 mM IMIP, or 90% polyethylene glycol: 10% DMSO vehicle, or 1 μM TPA (as a positive control for MVP release), or PDT using 20 J/cm² blue light. Immediately following PDT, the treated areas were given vehicle or imipramine. At 4 hours post-treatment, the mice were euthanized and MVP were measured from duplicate 6-mm punch biopsies and plasma. The data are mean±SD fold increase in MVP normalized to tissue weight/plasma volume. * Denotes statistically significant (P<0.05) changes from sham; #statistically significant (P<0.05) changes from comparator denoted by line by two-way ANOVA; NS not statistically significant.

FIG. 16, FIG. 17, and FIG. 18 show that imipramine was able to prevent an increase in MVP generation following PDT.

### Example 7: PDT-Induced MVP Formation is Dependent Upon aSMase

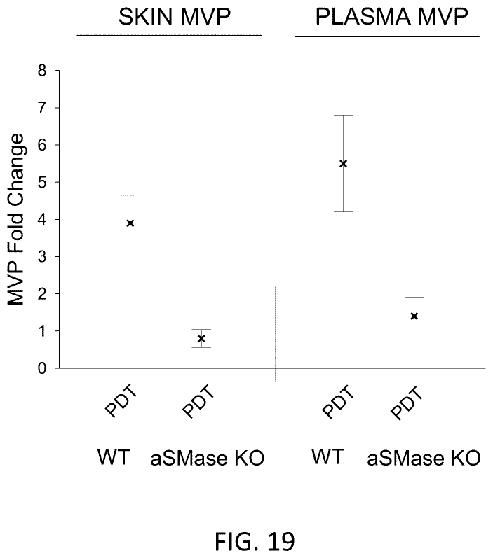

The ability of imipramine to block MVP formation in response to PDT suggests that this agent is acting via its ability to block aSMase. To confirm the role of aSMase in PDT-induced MVP generation, PDT effects were evaluated in the absence of aSMase was evaluated in a murine system. For the data shown in FIG. 19, wild-type or aSMase-deficient (Smpd1−/−) mice underwent sham or PDT using 20 J/cm² blue light. At 4 hours post-treatment, the mice were euthanized and MVP were measured from duplicate 6-mm punch biopsies and plasma. The data are mean±SD fold increase in MVP normalized to tissue weight/plasma volume. * Denotes statistically significant (P<0.05) changes from sham; NS not statistically significant.

FIG. 19 shows that the aSMase enzyme in mice was necessary for MVP production in response to topical PDT, validating this key MVP-producing enzyme as a target to block MVP-mediated pathologies.

### Example 8: Imipramine Reduces PDT-induced Immunosuppression

FIG. 20 and FIG. 21 show the role of aSMase in PDT-mediated inhibition of murine CHS reactions. For the data shown in FIG. 20, wild-type mice either were injected intraperitoneally with 250 ng CPAF as a positive control for immunosuppression, underwent PDT using 10 J/cm² blue light, or received no treatment (SHAM). Immediately thereafter, the back skin that underwent PDT was treated with 100 μL of 5% imipramine or 90% polyethylene glycol: 10% DMSO vehicle. Five days later the mice underwent sensitization with DNFB in PDT-treated (local immunosuppression) or non-PDT-treated skin (systemic immunosuppression). Nine days later the mice underwent elicitation reactions to ears with ear thickness measured at 24 hours. For the data shown in FIG. 21, wild type and aSMase-deficient (Smpd1−/−) mice underwent treatment with CPAF or PDT followed by DNFB sensitization/elicitation reactions as in FIG. 20. The data are mean±SD values. * Denotes statistically significant (P<0.05) changes from sham treatment values using two-way ANOVA.

FIG. 20 shows that topical PDT employing the neoantigen DNFB resulted in both local and systemic immunosuppressive effects in wild-type mice, and that topical imipramine given immediately after PDT, which blocks PDT-induced MVP release (see FIG. 19), blocks PDT-induced immunosuppression. Moreover, FIG. 21 shows that PDT of aSMase-deficient mice did not result in decreased systemic and local DNFB responses but treatment of mice lacking aSMase with CPAF resulted in systemic immunosuppressive CHS responses.

### Example 9: Localized UVB Irradiation of Human Subjects with Photosensitivity Results in Increased MVP Release but is Inhibited by Topical Imipramine

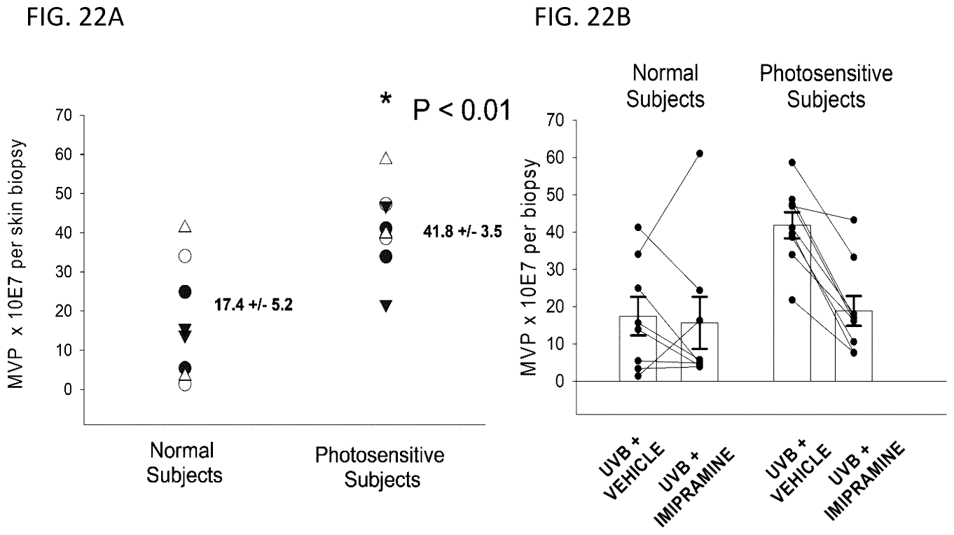

FIG. 22A compares normal subjects to subjects who have active photosensitivity disorders as denoted by positive screening. Both groups underwent a single treatment of 1 KJ/m² UVB on volar forearm, with addition of 4% imipramine or vehicle immediately after the irradiation. Four hours later, 5-mm punch biopsies were conducted of vehicle alone, imipramine alone, UVB+vehicle, and UVB+imipramine and MVP quantified. The individual data points are shown in FIG. 22A with the mean±SE of the groups for the UVB+vehicle for the normal and photosensitive subjects. FIG. 22B shows data for the UVB+vehicle versus UVB+imipramine for the two groups. These results suggest that UVB increases MVP in photosensitive subjects versus control subjects. Moreover, topical imipramine blocks the increased MVP release selectively in the photosensitive subjects.

### Example 10: UVR Treatment of XPA KO Mice Results in Increased MVP Release, Dependent Upon the PAFR

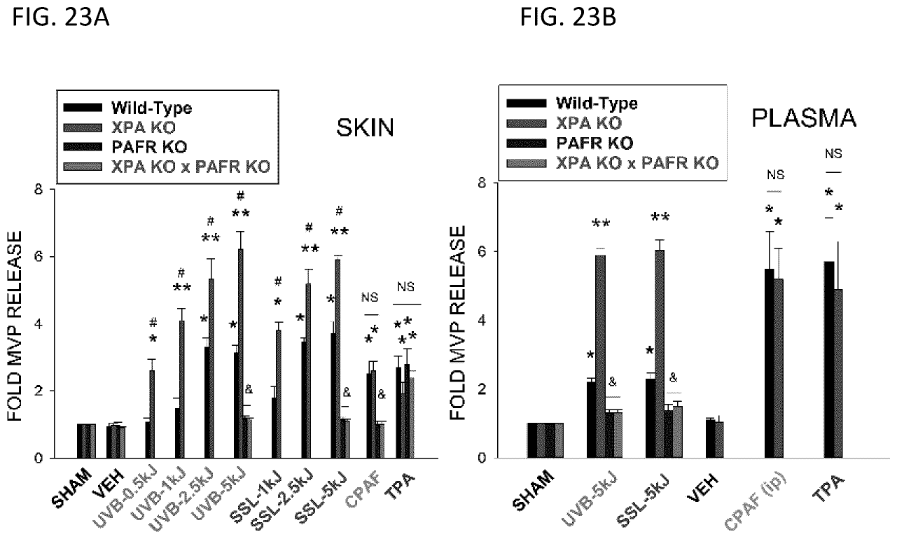

Groups of at least eight wild-type, XPA KO (Xpa−/−), PAFR KO (Ptafr−/−), or mice deficient in both XPA and PAFR (XPA KO×PAFR KO) were treated with various fluences of UVB or SSL, no treatment (sham), 100 μL of Platelet-activating factor agonist CPAF (100 μM) or phorbol ester TPA (100 μM) or DMSO:ethanol (90:10) vehicle. Four hours later duplicate skin biopsies were obtained, and MVP quantified, with the results shown in FIG. 23A. The procedures were repeated with the exception that CPAF was provided by 250 ng intraperitoneal (i.p.) injection and blood was obtained, and MVP quantified, with the results shown in FIG. 23B. The data are mean±SE fold MVP release from sham-treated mice. Statistically significant *(P<0.05), **(P<0.01) changes from sham or vehicle values. Statistically significant (P<0.05) changes from the #XPA KO vs Wild-type and & PAFR KO vs Wild-type or XPA KO mice. N.S., not statistically significant.

These results clearly demonstrate that mice that lack XPA whose phenotype include photosensitivity generate increased MVP. Of interest, a keratinocyte-derived cell line deficient in XPA (HaCaT XPA KO) also generates increased MVP, which is blocked by imipramine. XPA KO mice given imipramine post-UVB exhibit decreased erythema and decreased cytokine expression levels.

### Example 11: Topical Imipramine Blocks the Erythema Response of Low-Fluence UVB on Subjects with Rosacea

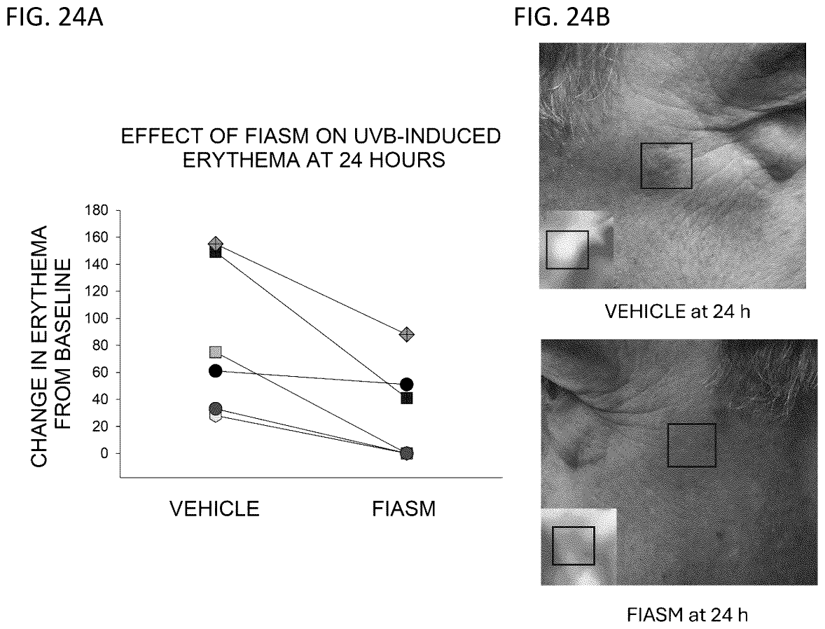

Adult subjects underwent treatment with either 0.1 ml of 4% imipramine or polyethylene glycol: DMSO (90:10) vehicle to small 2.5×2.5 cm areas of temple skin. 30 minutes later, a 1 cm area of previously treated skin was irradiated with a low 300 J/m² UVB. Erythema was measured before UVB and at various times post-UVB irradiation in blinded fashion with a mexameter and temperature using a thermal imaging camera. FIG. 24A show the resulting data for six subjects in which one temple was treated with imipramine+UVB and the other temple treated with vehicle+UVB. Values represent the change in erythema from baseline for each subject at 24 hours post-UVB. FIG. 24B shows an example of one subject demonstrating erythema responses noted on vehicle+UVB-treated temple (top picture) versus imipramine+UVB (bottom picture). The boxes represent the area treated with UVB. Insets into the photographs are thermal camera images of the UVB-treated skin, with white areas on the thermal imaging camera denoting increased temperature. Note that there is less erythema and less temperature signature in the imipramine+UVB versus vehicle+UVB treated skin.

The skin protective composition s and methods of the present disclosure can comprise, consist of, or consist essentially of the essential elements and limitations of the disclosure described herein, as well as any additional or optional ingredients, components, or limitations described herein or otherwise useful. The terms “comprising,” “having,” and “including” are used in their open, non-limiting sense. The expression “inclusive” for a range of concentrations means that the limits of the range are included in the defined interval.

Other than in the operating examples, or where otherwise indicated, all numbers expressing quantities of ingredients and/or reaction conditions are to be understood as being modified in all instances by the term “about,” meaning within +/−5%, 4%, 3%, 2%, or 1% of the indicated number. Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

All percentages, parts and ratios herein are relative to the amount of active agent, based upon the total weight of the hair refreshing composition of the present disclosure, unless otherwise indicated.

As used herein, the terms “free” and “essentially free” and “exclude/excludes” are intended to denote that the component is absent entirely from the hair refreshing composition or is present in an amount considered by those skilled in the art to not provide an effect on the hair refreshing composition. For example, the component may be present in an amount below the level of detection or may be present in an amount less than 3%, less than 2%, less than 1%, less than 0.5%, less than 0.1%, less than 0.01%, or less than 0.001%.

It should also be understood that the precise numerical values used in the specification and claims form additional embodiments of the disclosure and are intended to include any ranges which may be narrowed to any two end points disclosed within the exemplary ranges and values provided, as well as the specific end points themselves. Efforts have been made to ensure the accuracy of the numerical values disclosed herein. Any measured numerical value, however, can inherently contain certain errors resulting from the standard deviation found in its respective measuring technique. All percentages, parts and ratios herein are based upon the total weight of the compositions of the present disclosure, unless otherwise indicated.

All ranges and values disclosed herein are inclusive and combinable. For examples, any value or point described herein that falls within a range described herein can serve as a minimum or maximum value to derive a sub-range, etc. Furthermore, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc.

As used herein, the expression “at least one” is interchangeable with the expression “one or more” and thus includes individual components as well as mixtures/combinations. The terms “a” and “the” are understood to encompass the plural as well as the singular.

The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

The term “INCI” is an abbreviation of International Nomenclature of Cosmetic Ingredients, which is a system of names provided by the International Nomenclature Committee of the Personal Care Products Council to describe personal care ingredients.

The term “weight ratio” or “mass ratio” as used herein, references the amount of a substance in proportion to a mixture containing said substance, and is calculated by dividing the amount of said substance by weight contained in the mixture by the weight of the mixture containing said substance. As an example, a weight ratio of 0.4 for substance A in a mixture of A, B, and C indicates that the weight of substance A divided by the total weight of substances A, B, and C is 0.4.

All publications and patent applications cited in this specification are herein incorporated by reference, and for any and all purposes, as if each individual publication or patent application were specifically and individually indicated to be incorporated by reference. In the event of an inconsistency between the present disclosure and any publications or patent application incorporated herein by reference, the present disclosure controls.

What is claimed:

1. A skin protective composition for topical application to keratinous tissue comprising:
a. a functional inhibitor of acid sphingomyelinase (FIASM); and
b. a dermatologically acceptable carrier.

2. The skin protective composition according to claim 1, wherein the FIASM is a compound selected from the group consisting of amineptine, amitriptyline, amitripylinoxide, amoxapine, butriptyline, clomipramine, demexiptiline, desipramine, dibenzepin, dimetacrine, dosulepin, doxepine, imipramine, imipraminoxide, iprindole, lofepramine, melitracen, metapramine, nitroxazepine, nortriptyline, noxiptiline, pipofezine, propizenpine, protriptyline, quinupramine, tianeptine, trimipramine, and combinations thereof.

3. The skin protective composition according to claim 1, wherein the FIASM is present at a concentration in a range from about 0.1% to about 5% by weight based on the weight of the skin protective composition.

4. The skin protective composition according to claim 1, the carrier comprising at least one dermal penetrating enhancer selected from the group consisting of microemulsions, alcohols, polyols, surfactants, fatty acids, amines, amides, terpenes, sulfoxides, esters, and combinations thereof.

5. The skin protective composition according to claim 1, wherein the FIASM comprises imipramine present in a range from about 2% to about 5% by weight based on the weight of the skin protective composition, and the dermatologically acceptable carrier comprises propylene glycol present in a range from about 1% to about 25% by weight based on the weight of the skin protective composition.

6. The skin protective composition according to claim 1, comprising at least one additive selected from the group consisting of vitamins, fragrances, dyes, preservatives, oils, essential oils, salts, neutralizing or pH-adjusting agents, and combinations thereof.

7. The skin protective composition according to claim 1, further comprising at least one UV filter selected from the group consisting of titanium dioxide; zinc oxide; iron oxides; cerium oxides; zirconium oxides; anthranilic compounds; dibenzoylmethane compounds; cinnamic compounds; salicylic compounds; camphor compounds; benzophenone compounds; diphenylacrylate compounds; triazine compounds; benzotriazole compounds; benzalmalonate compounds; benzimidazole compounds; imidazoline compounds; bis-benzoazolyl compounds; p-aminobenzoic acid (PABA) compounds; methylenebis(hydroxyphenylbenzotriazole) compounds; benzoxazole compounds; dimers derived from α-alkylstyrene; 4,4-diarylbutadienes compounds; guaiazulene and derivatives thereof; rutin and derivatives thereof; flavonoids; bioflavonoids; oryzanol and derivatives thereof; quinic acid and derivatives thereof; and combinations thereof.

8. The skin protective composition according to claim 1, wherein the skin protective composition is a cream, a gel, an ointment, or a spray, and is anhydrous, a water-in-oil emulsion, or an oil-in-water emulsion.

9. A method of ameliorating a skin condition in a subject, the method comprising: topically administering to a subject a skin protective composition comprising a functional inhibitor of acid sphingomyelinase (FIASM), the FIASM being present in a dermatologically acceptable carrier comprising one or a combination of ingredients selected from the group consisting of water, water-based solvents, oils, oil-based solvents, antimicrobials, antibiotics, redness reducers, antioxidants, emollients, and combinations thereof.

10. The method according to claim 9, wherein the FIASM is a compound selected from the group consisting of amineptine, amitriptyline, amitripylinoxide, amoxapine, butriptyline, clomipramine, demexiptiline, desipramine, dibenzepin, dimetacrine, dosulepin, doxepine, imipramine, imipraminoxide, iprindole, lofepramine, melitracen, metapramine, nitroxazepine, nortriptyline, noxiptiline, pipofezine, propizenpine, protriptyline, quinupramine, tianeptine, trimipramine, and combinations thereof.

11. The method according to claim 9, wherein the FIASM is present at a concentration in a range from about 0.1% to about 5% by weight based on the weight of the skin protective composition.

12. The method according to claim 9, wherein the FIASM comprises imipramine present in a range from about 0.1% to about 5% by weight based on the weight of the skin protective composition, and the dermatologically acceptable carrier comprises propylene glycol present in a range from about 1% to about 25% by weight based on the weight of the skin protective composition.

13. The method according to claim 9, further comprising administering the skin protective composition at a frequency of at least once prior to exposure to sun, after exposure to sun, or both.

14. A skin care system for topical application to keratinous tissue comprising:
a. a skin protective component comprising (i) a functional inhibitor of acid sphingomyelinase (FIASM); and (ii) a dermatologically acceptable carrier; and
b. a photosensitizing component comprising at least one photosensitizer.

15. The skin care system according to claim 14, wherein the FIASM is a compound selected from the group consisting of amineptine, amitriptyline, amitripylinoxide, amoxapine, butriptyline, clomipramine, demexiptiline, desipramine, dibenzepin, dimetacrine, dosulepin, doxepine, imipramine, imipraminoxide, iprindole, lofepramine, melitracen, metapramine, nitroxazepine, nortriptyline, noxiptiline, pipofezine, propizenpine, protriptyline, quinupramine, tianeptine, trimipramine, and combinations thereof.

16. The skin care system according to claim 14, wherein the FIASM is present at a concentration in a range from about 0.1% to about 5% by weight based on the weight of the skin protective composition.

17. The skin care system according to claim 14, the carrier comprising at least one dermal penetrating enhancer selected from the group consisting of microemulsions, alcohols, polyols, surfactants, fatty acids, amines, amides, terpenes, sulfoxides, esters, and combinations of these.

18. The skin care system according to claim 14, wherein the FIASM comprises imipramine present in a range from about 2% to about 5% by weight based on the weight of the skin protective composition, and the dermatologically acceptable carrier comprises propylene glycol present in a range from about 1% to about 25% by weight based on the weight of the skin protective composition.

19. The skin care system according to claim 14, further comprising at least one UV filter selected from the group consisting of titanium dioxide; zinc oxide; iron oxides; cerium oxides; zirconium oxides; anthranilic compounds; dibenzoylmethane compounds; cinnamic compounds; salicylic compounds; camphor compounds; benzophenone compounds; diphenylacrylate compounds; triazine compounds; benzotriazole compounds; benzalmalonate compounds; benzimidazole compounds; imidazoline compounds; bis-benzoazolyl compounds; p-aminobenzoic acid (PABA) compounds; methylenebis(hydroxyphenylbenzotriazole) compounds; benzoxazole compounds; dimers derived from α-alkylstyrene; 4,4-diarylbutadienes compounds; guaiazulene and derivatives thereof; rutin and derivatives thereof; flavonoids; bioflavonoids; oryzanol and derivatives thereof; quinic acid and derivatives thereof; and combinations thereof.

20. The skin care system according to claim 14, wherein the at least one photosensitizer is selected from the group consisting of aminolevulinic acid, methyl aminolevulinate, 5-aminileuvulinic acid, porphyrin, protoporfin IX, purlytin, verteporfin, HPPH, temoporfin, methylene blue, photofrin, hematoporphyrin, Talaporfin, benzopophyrin derivative monoacid, Lutetium texaphyrin, metallophthalocyanine, metallo-naphthocyaninesulfobenzo-porphyrazine, metallo-naphthalocyanines, zinc tetrasulfophthalocyanine, bacteriochlorins, metallochlorins, chlorine derivative, Tetra(m-hydroxyphenyl)chlorin (mTHPC), pheophorbide, dibromofluorescein (DBF), IR700DX, naphthalocyanine, porphyrin derivative, and combinations thereof.
